(19) United States
(12) Patent Application Publication
(10) Pub. No.: US 2026/0290638 A1
(43) Pub. Date: Sep. 24, 2026

(54) MANUFACTURING SYSTEMS AND METHODS

(71) Applicant: Novartis AG, Basel (CH)

(72) Inventors: Graham Stewart Watters, East Hanover, NJ (US); Oner Tuyluoglu, East Hanover, NJ (US); Ian McLaurin, East Hanover, NJ (US)

(21) Appl. No.: 19/572,347

(22) Filed: Mar. 19, 2026

Related U.S. Application Data
(60) Provisional application No. 63/775,932, filed on Mar. 21, 2025.
(60) Provisional application No. 63/855,768, filed on Aug. 1, 2025.
(60) Provisional application No. 63/981,256, filed on Feb. 12, 2026.

(51) Int. Cl. G21F 5/14; A61J 3/00; G21F 5/015; G21F 5/02; G21F 5/12

(52) U.S. Cl. CPC G21F 5/14; A61J 3/00; G21F 5/015; G21F 5/02; G21F 5/125

(57) ABSTRACT

A method of manufacture includes synthesizing a radiopharmaceutical composition in a first unit. The method further includes packaging the radiopharmaceutical composition into a radiopharmaceutical product in a second unit. A modular manufacturing system includes a first unit to synthesize a radiopharmaceutical composition. The modular manufacturing system further includes a second unit to package the radio pharmaceutical composition into a radiopharmaceutical product.

### CROSS-REFERENCE TO RELATED APPLICATIONS

The present application claims priority to U.S. Provisional Patent Application No. 63/775,932, filed on Mar. 21, 2025, U.S. Provisional Patent Application No. 63/855,768, filed on Aug. 1, 2025, and U.S. Provisional Patent Application No. 63/981,256, filed on Feb. 12, 2026, the contents of which are incorporated herein by reference in their entireties.

### BACKGROUND

Due to the radioactivity of radiopharmaceutical drug products, their manufacture is often carried out in shielded units. The current practice is to perform the entire production of a radiopharmaceutical product in a single shielded isolator that allows manipulations via gloves or gripping devices from outside the isolator and material locks for educt and product transfers into and out of said isolator. The isolator must satisfy certain cleanliness zoning criteria for aseptic production of a sterile radiopharmaceutical product. There remains a need for versatile systems and methods, providing increased efficiency, for the manufacture of radiopharmaceuticals.

### SUMMARY

In view of the foregoing, the Inventors have recognized and appreciated the advantages of the systems and methods as described herein.
Implementation 1: A method of manufacturing, comprising: synthesizing a radiopharmaceutical composition in a first unit; and packaging the radiopharmaceutical composition into a radiopharmaceutical product in a second unit.
Implementation 2: The method of Implementation 1, further comprising maintaining the first unit and the second unit in different environments.
Implementation 3: The method of any of Implementations 1-2, further comprising maintaining the first unit in a Grade D environment.
Implementation 4: The method of any of Implementations 1-3, further comprising maintaining the second unit in a Grade C environment.
Implementation 5: The method of any of Implementations 1-4, wherein the synthesizing further comprises compounding the radiopharmaceutical composition.
Implementation 6: The method of any of Implementations 1-5, wherein the synthesizing further comprises cold and hot dilution manufacturing of the radiopharmaceutical composition in the first unit.
Implementation 7: The method of any of Implementations 1-6, wherein the packaging further comprises sanitizing the radiopharmaceutical product in the second unit.
Implementation 8: The method of any of Implementations 1-7, wherein the packaging further comprises dispensing, using an automated process, the radiopharmaceutical composition into a packaging material to form the radiopharmaceutical product of a predetermined dosage.
Implementation 9: The method of any of Implementations 1-8, wherein the packaging further comprises using disposable cassettes to carry the radiopharmaceutical composition and the radiopharmaceutical product in the first unit and second unit, respectively.
Implementation 10: The method of any of Implementations 1-9, wherein the packaging further comprises dispensing at least one radionuclide as part of forming the radiopharmaceutical product in the second unit.
Implementation 11: The method of any of Implementations 1-10, further comprising controlling flow properties of a fluid comprising the synthesized radiopharmaceutical composition before the fluid enters the second unit.
Implementation 12: The method of any of Implementations 1-11, further comprising transporting the radiopharmaceutical composition from the first unit to the second unit via a transport machine.
Implementation 13: The method of Implementation 12, the transport machine comprising an autonomous guided vehicle or an autonomous mobile robot.
Implementation 14: The method of any of Implementations 12-13, the first unit including a shielded enclosure and a rapid transfer port providing access through the shielded enclosure of the first unit, the transport machine retrieving the radiopharmaceutical composition from the first unit via the rapid transfer port of the first unit.
Implementation 15: The method of any of Implementations 12-14, the second unit including a shielded enclosure and a rapid transfer port providing access through the shielded enclosure of the second unit, the transport machine delivering the radiopharmaceutical composition to the second unit via the rapid transfer port of the second unit.
Implementation 16: The method of any of Implementations 12-15, the transport machine traveling through a shielded enclosure while transporting the radiopharmaceutical composition from the first unit to the second unit via a transport machine.
Implementation 17: The method of any of Implementations 1-16, the step of packaging the radiopharmaceutical composition into the radiopharmaceutical product in the second unit including: automatically dispensing the radiopharmaceutical composition into a vessel, and automatically inserting a stopper into the vessel.
Implementation 18: The method of Implementation 17, the vessel comprising a vial or a syringe barrel.
Implementation 19: The method of any of Implementations 17-18, further including placing the vessel in a shield assembly.
Implementation 20: The method of Implementation 19, the step of placing the vessel in a shield assembly being performed in the second unit.
Implementation 21: The method of any of Implementations 1-20, further comprising performing visual inspection of the packaged radiopharmaceutical composition.
Implementation 22: The method of Implementation 21, the step of performing visual inspection of the packaged radiopharmaceutical composition being performed in a third unit, the method further comprising transporting the packaged radiopharmaceutical composition from the second unit to the third unit.
Implementation 23: The method of any of Implementations 1-22, further comprising testing a closure integrity of the packaged radiopharmaceutical composition.
Implementation 24: The method of Implementation 23, the step of testing a closure integrity of the packaged radiopharmaceutical composition being performed in a third unit, the method further comprising transporting the packaged radiopharmaceutical composition from the second unit to the third unit.
Implementation 25: The method of any of Implementations 1-24, further comprising applying a label to the packaged radiopharmaceutical composition, the label including information associated with manufacture of the radiopharmaceutical composition.
Implementation 26: The method of Implementation 25, the step of applying a label to the packaged radiopharmaceutical composition being performed in a third unit, the method further comprising transporting the packaged radiopharmaceutical composition from the second unit to the third unit.
Implementation 27: The method of any of Implementations 1-26, further comprising providing a dose calibration of the radiopharmaceutical composition.
Implementation 28: The method of Implementation 27, the step of providing a dose calibration of the radiopharmaceutical composition being performed in the first unit.
Implementation 29: The method of any of Implementations 27-28, the step of providing a dose calibration of the radiopharmaceutical composition being performed in a third unit, the method further comprising transporting the packaged radiopharmaceutical composition from the second unit to the third unit.
Implementation 30: A modular manufacturing system, comprising: a first unit to synthesize a radiopharmaceutical composition; and a second unit to package the radio pharmaceutical composition into a radiopharmaceutical product.
Implementation 31: The modular manufacturing system of Implementation 30, wherein the first unit has a Grade D environment.
Implementation 32: The modular manufacturing system of any of Implementations 30-31, wherein the second unit has a Grade C environment.
Implementation 33: The modular manufacturing system of any of Implementations 30-32, wherein the first unit further comprises a synthesis module that is configured to synthesize the radiopharmaceutical composition.
Implementation 34: The modular manufacturing system of any of Implementations 30-33, wherein the second unit further comprises a dispensing module that is configured to dispense the radiopharmaceutical composition using an automated process, and a sanitizing unit that is configured to sanitize radiopharmaceutical product.
Implementation 35: The modular manufacturing system of any of Implementations 30-34, wherein the first unit further comprises a synthesis module that is configured to synthesize the radiopharmaceutical composition; the second unit further comprises a dispensing module that is configured to dispense the radiopharmaceutical composition using an automated process, and a sanitizing unit that is configured to sanitize radiopharmaceutical product; and the first unit, the second unit, or both, further comprises a shield enclosing, respectively the synthesis module in the first unit and the dispensing module and the sanitizing unit in the second unit.
Implementation 36: The modular manufacturing system of any of Implementations 30-35, wherein the first unit and second unit are to receive disposable cassettes carrying the radiopharmaceutical composition and the radiopharmaceutical product, respectively.
Implementation 37: The modular manufacturing system of any of Implementations 30-36, further comprising a flow control module that is configured to regulate flow properties of a fluid comprising the synthesized radiopharmaceutical composition before the fluid enters the second unit.
Implementation 38: The modular manufacturing system of any of Implementations 30-37, further comprising a flow control module comprising a pump, a valve, or a combination thereof, to regulate, using an automated process, flow properties of a fluid comprising the synthesized radiopharmaceutical composition before the fluid enters the second unit.
Implementation 39: The modular manufacturing system of any of Implementations 30-38, wherein the second unit further comprises a plurality of cells dedicated to dispensing radiopharmaceutical product and radionuclides.
Implementation 40: The modular manufacturing system of any of Implementations 30-38, further comprising one or more transport machines operable to transport the radiopharmaceutical composition from the first unit to the second unit.
Implementation 41: The modular manufacturing system of Implementation 40, the one or more transport machines comprising an autonomous guided vehicle or an autonomous mobile robot.
Implementation 42: The modular manufacturing system of any of Implementations 40-41, the first unit including a shielded enclosure and a rapid transfer port providing access through the shielded enclosure of the first unit, one or more transport machines being operable to retrieve the radiopharmaceutical composition from the first unit via the rapid transfer port of the first unit.
Implementation 43: The modular manufacturing system of any of Implementations 40-42, the second unit including a shielded enclosure and a rapid transfer port providing access through the shielded enclosure of the second unit, one or more transport machines being operable to deliver the radiopharmaceutical composition to the second unit via the rapid transfer port of the second unit.
Implementation 44: The modular manufacturing system of any of Implementations 40-43, further comprising a shielded enclosure, one or more transport machines being operable to traveling through the shielded enclosure while transporting the radiopharmaceutical composition from the first unit to the second unit via a transport machine.
Implementation 45: The modular manufacturing system of any of Implementations 30-44, the second unit being operable to package the radiopharmaceutical composition into the radiopharmaceutical product by: automatically dispensing the radiopharmaceutical composition into a vessel, and automatically inserting a stopper into the vessel.
Implementation 46: The modular manufacturing system of Implementation 45, the vessel comprising a vial or a syringe barrel.
Implementation 47: The modular manufacturing system of any of Implementations 45-46, further comprising an assembly stage operable to place the vessel in a shield assembly.
Implementation 48: The modular manufacturing system of Implementation 47, the assembly stage being positioned in the second unit.
Implementation 49: The modular manufacturing system of any of Implementations 30-48, further comprising a visual inspection stage operable to provide visual inspection of the one or more transport machines radiopharmaceutical composition.
Implementation 50: The modular manufacturing system of Implementation 49, the visual inspection stage being positioned in a third unit, separate from the first unit and the second unit.
Implementation 51: The modular manufacturing system of any of Implementations 30-50, further comprising a closed container integrity tester (CCIT) stage, the CCIT stage being operable to test a closure integrity of the packaged radiopharmaceutical composition.
Implementation 52: The modular manufacturing system of Implementation 51, the CCIT stage being positioned in a third unit, separate from the first unit and the second unit.
Implementation 53: The modular manufacturing system of any of Implementations 30-52, further comprising a labeling stage operable to apply a label to the packaged radiopharmaceutical composition, the label including information associated with the manufacture of the radiopharmaceutical composition.
Implementation 54: The modular manufacturing system of Implementation 53, the labeling stage being positioned in a third unit, separate from the first unit and the second unit.
Implementation 55: The modular manufacturing system of any of Implementations 30-54, further comprising a dose calibration stage operable to provide a dose calibration of the radiopharmaceutical composition.
Implementation 56: The modular manufacturing system of Implementations 55, the dose calibration stage being positioned in the first unit.
Implementation 57: The modular manufacturing system of any of Implementations 55-56, the dose calibration stage being positioned in a third unit, separate from the first unit and the second unit.
Implementation 58: The modular manufacturing system of Implementation 30, further comprising a transport machine to transport the radiopharmaceutical composition from the first unit to the second unit, wherein the first and second unit and the transport machine are disposed in an external environment.
Implementation 59: The modular manufacturing system of Implementation 58, further comprising a radiation shielded container having a bulk solution container disposed therein, the bulk solution container containing the radiopharmaceutical composition, wherein the transport machine transports the radiation shielded container having the bulk solution container disposed therein between the first unit and the second unit.
Implementation 60: The modular manufacturing system of any of Implementations 58 to 59, wherein the transport machine further comprises a mechanism for raising and lowering the radiation shielded container.
Implementation 61: The modular manufacturing system of any of Implementations 58 to 60, the first unit further comprising an isolator having a synthesis module disposed therein; a first decontamination module; a first valve coupling the isolator to the first decontamination module, wherein the first valve, when opened, fluidly couples an interior volume of the isolator to an interior volume of the first decontamination module; and a second valve disposed in the first decontamination module, wherein the second valve, when opened, fluidly couples the interior volume of the first decontamination module to the external environment.
Implementation 62: The modular manufacturing system of any of Implementations 58 to 61, the second unit further comprising a dispensing module; a second decontamination module; a third valve coupling the second unit to the second decontamination module, wherein the third valve, when opened, fluidly couples an interior volume of the second module to an interior volume of the second decontamination module; and a fourth valve disposed in the second decontamination module, wherein the fourth valve, when opened, fluidly couples the intervolume of the second decontamination module to the external environment.
Implementation 63: The modular manufacturing system of any of Implementations 58 to 60, wherein the dispensing module further comprises a radiation shielded pocket configured to interface with the radiation shieled container, wherein the radiation shielded pocket covering one or more openings in the dispensing module, and wherein the one or more openings is configured to receive the radiopharmaceutical composition from the bulk solution container.
Implementation 64: The modular manufacturing system of Implementation 63, wherein the radiation shielded pocket further comprises a housing having an interior volume, where the interior volume of the housing is fluidly coupled to a lower opening and an upper opening, wherein the one or more openings are fluidly coupled to the interior volume of the housing between the lower and upper openings; and a lid coupled to the housing and movable between an open position and a closed position, wherein the lid covers the upper opening when in a closed position, wherein the radiation shielded container is configured to enter the interior volume of the housing through the lower opening.
Implementation 65: The modular manufacturing system of any of Implementations 59 to 64, wherein the radiation shielded container further comprises a container portion having an interior volume and a first opening; and a lid removably coupled to the container portion to cover the first opening and enclose the interior volume, wherein the bulk solution container is disposed in the interior volume of the container portion.
Implementation 66: The modular manufacturing system of Implementation 65, wherein the container portion further comprises a second opening fluidly coupled to the interior volume of the container portion, the second opening for accessing the bulk solution container
Implementation 67: The modular manufacturing system of Implementation 65, wherein the radiation shielded container further comprises a secondary lid covering the first opening and disposed between the lid and the container portion, wherein the secondary lid comprises a third opening fluidly coupled to the interior volume of the container portion.
Implementation 68: The modular manufacturing system of any of claims 59 to 67, wherein the bulk solution container further comprises a bulk solution container portion for holding the radiopharmaceutical composition; and an outlet connector coupled to the bulk solution container portion.
Implementation 69: The modular manufacturing system of Implementation 68, wherein the outlet connector of the bulk solution container is disposed through the second opening.
Implementation 70: The modular manufacturing system of any of Implementations 68 to 69, wherein the bulk solution container further comprises a backup outlet connector coupled to the bulk solution container portion.
Implementation 71: The modular manufacturing system of any of Implementations 68 to 70, wherein the bulk solution container further comprises an inlet connector coupled to the bulk solution container portion.
Implementation 72: The modular manufacturing system of any of Implementations 68 to 71, wherein the bulk solution container further comprises a vent connector coupled to the bulk solution container portion.
Implementation 73: The modular manufacturing system of any of Implementations 58 to 72, further comprising a transfer kit for transferring the radiopharmaceutical composition from the bulk solution container into the second module.
Implementation 74: The modular manufacturing system of Implementation 73, wherein the transfer kit further comprises a tube having a first connector at one end of the tube and a second connector at an opposing end of the tube, wherein the first connector is configured to be coupled to the bulk solution container.
Implementation 75: The modular manufacturing system of Implementation 73, wherein the transfer kit further comprises one or more filters for decontaminating the radiopharmaceutical composition upon exit from the bulk solution container, wherein the one more filters are disposed in-line in the tube.
Implementation 76: The modular manufacturing system of Implementation 75, further comprising an auxiliary line coupled to a vent connector disposed on the one or more filters.
Implementation 77: The modular manufacturing system of Implementation 74, further comprising a peristaltic pump coupled to the tube of the transfer kit to facilitate movement of the radiopharmaceutical composition therethrough.
Implementation 78. The modular manufacturing system of Implementation 73, wherein the transfer kit further comprises a syringe; a valve coupled to the syringe; a first rigid tube having a first end coupled to the valve; and a second rigid tube having a first end coupled to the valve, wherein a second end of the first rigid tube is configured to be coupled to the bulk solution container, wherein the valve, in a first open position, permits fluid flow between the first rigid tube and the syringe, and no fluid flow between the first rigid tube and the second rigid tube, and between the syringe and the second rigid tube, and wherein the valve, in a second open position, permits fluid flow between the syringe and the second rigid tube, and no fluid flow between the first rigid tube and the second rigid tube, and between the syringe and the first rigid tube.
Implementation 79: The modular manufacturing system of Implementation 77, wherein the transfer kit further comprises one or more filters for decontaminating the radiopharmaceutical composition upon exit from the bulk solution container, wherein the one more filters are disposed in-line in at least one of the first rigid tube or the second rigid tube.
Implementation 80: The method of Implementation 1, further comprising transporting the radiopharmaceutical composition synthesized in the first unit to the second unit for packaging.
Implementation 81: The method of Implementation 80, further comprising loading the radiopharmaceutical composition synthesized in the first unit into a bulk solution container.
Implementation 82: The method of Implementation 81, wherein the bulk solution container further comprises a bulk solution container portion for holding the radiopharmaceutical composition; an outlet connector coupled to the bulk solution container portion; and an inlet connector coupled to the bulk solution container portion.
Implementation 83: The method of Implementation 82, wherein the bulk solution container further comprises a backup outlet connector coupled to the bulk solution container portion.
Implementation 84: The method of any of Implementations 82 to 83, wherein the bulk solution container further comprises a vent connector coupled to the bulk solution container portion.
Implementation 85: The method of any of Implementations 82 to 84, wherein loading the radiopharmaceutical composition further comprises loading the radiopharmaceutical composition through the inlet connector into the bulk solution container portion.
Implementation 86: The method of any of Implementations 82 to 85, wherein the bulk solution container is disposed in a radiation shielded container, the radiation shielded container comprising a container portion having an interior volume and a first opening; and a lid removably coupled to the container portion to cover the first opening and enclose the interior volume, wherein the bulk solution container is disposed in the interior volume of the container portion.
Implementation 87: The method of Implementation 86, wherein the container portion further comprises a second opening fluidly coupled to the interior volume of the container portion, the second opening for accessing the bulk solution container disposed in the interior volume of the container portion.
Implementation 88: The method of Implementation 86, wherein the radiation shielded container further comprises a secondary lid covering the first opening and disposed between the lid and the container portion, wherein the secondary lid comprises a third opening fluidly coupled to the interior volume of the container portion.
Implementation 89: The method of any of Implementations 87 to 88, wherein the radiation shielded container having the bulk solution container disposed therein, the bulk solution container containing the radiopharmaceutical composition, is disposed in an interior volume of a isolator the first unit, the isolator having a synthesis module disposed in the interior volume thereof.
Implementation 90: The method of Implementation 89, further comprising opening a first valve separating the interior volume of the isolator of from an interior volume of a first decontamination module, the first decontamination module coupled to the isolator through the first valve; transferring the radiation shielded container from the interior volume of the isolator to the interior volume of the first decontamination module; and closing the first valve to separate the interior volume of the isolator from the interior volume of the first decontamination module.
Implementation 91: The method of Implementation 90, further comprising decontaminating the radiation shielded container in the first decontamination module.
Implementation 92: The method of any of Implementations 90 to 91, further comprising opening a second valve separating the interior volume of the first decontamination module from an external environment; removing the radiation shielded container from the interior volume of the first decontamination module; and loading the radiation shielded container onto a transportation machine.
Implementation 93: The method of any of Implementations 87 to 88, wherein the radiation shielded container having the bulk solution container disposed therein is disposed in an external environment, wherein the external environment is separated from the interior volume of an isolator the first unit, the isolator having a synthesis module disposed in the interior volume thereof.
Implementation 94: The method of Implementation 93, wherein loading the radiopharmaceutical composition further comprises coupling the inlet connector of the bulk solution container to a corresponding connector of a tube of a transfer kit extending through an opening in the isolator, the opening extending between the interior volume of isolator and the external environment; and loading the radiopharmaceutical composition from the interior volume of the isolator into the bulk solution container portion of the bulk solution container.
Implementation 95: The method of Implementation 94, wherein the radiation shielded container is disposed on a transportation machine in the external environment while loading the radiopharmaceutical composition from the interior volume of the isolator, or loading the radiation shielded container onto a transport machine after loading the radiopharmaceutical composition from the interior volume of the isolator.
Implementation 96: The method of Implementation 92 or 95, further comprising moving the transport machine to the second unit.
Implementation 97: The method of Implementation 96, further comprising closing a third valve separating an interior volume of a dispensing module of the second unit from an interior volume of a second decontamination module, the second decontamination module coupled to the second unit through the third valve; opening a fourth valve separating the interior volume of the second decontamination module from the external environment; transferring the radiation shielded container from the external environment to the interior volume of the second decontamination module; and closing the fourth valve to separate the interior volume of the second decontamination module from the external environment.
Implementation 98: The method of Implementation 97, further comprising decontaminating the radiation shielded container in the second decontamination module.
Implementation 99: The method of Implementation 98, further comprising opening the third valve to fluidly couple the interior volume of the dispensing module to the interior volume of the second decontamination chamber; and transferring the decontaminated radiation shielded container to the interior volume of the dispensing module.
Implementation 100: The method of Implementation 99, further comprising coupling the bulk solution container to a transfer kit to transfer the radiopharmaceutical composition from the bulk solution container.
Implementation 101: The method of Implementation 100, wherein the transfer kit further comprises a tube having a first connector at one end of the tube and a second connector at an opposing end of the tube, and wherein coupling the bulk solution container to the transfer kit further comprises coupling the outlet connector of the bulk solution container to the first connector of the transfer kit; moving the radiopharmaceutical composition from the bulk solution container portion through the tube of the transfer kit using a peristaltic pump; and filtering the radiopharmaceutical composition between entry of the radiopharmaceutical composition into the first flexible tube and the exit of the radiopharmaceutical composition from the second end of the second flexible tube.
Implementation 102: The method of Implementation 101, wherein the transfer kit further comprises one or more filters, wherein the one more filters are disposed in-line in the tube.
Implementation 103: The method of Implementation 93, wherein the transfer kit further comprises a syringe; a valve coupled to the syringe; a first rigid tube having a first end coupled to the valve and a second end opposite the first end; and a second rigid tube having a first end coupled to the valve and a second end opposite the first end, and wherein coupling the bulk solution container to the transfer kit further comprises coupling the outlet connector of the bulk solution container to the second end of the first rigid tube of the transfer kit; moving the valve to a first open position where the first rigid tube is open to the syringe and the second rigid tube is closed to the syringe; pulling a plunger of the syringe to move the radiopharmaceutical composition from the bulk solution container portion into the syringe through the first rigid tube; moving the valve to a second open position where the second rigid tube is open to the syringe and the first rigid tube is closed to the syringe; pushing the plunger of the syringe to move the radiopharmaceutical composition from the syringe into the second flexible tube; and filtering the radiopharmaceutical composition between entry of the radiopharmaceutical composition into the first rigid tube and the exit of the radiopharmaceutical composition from the second end of the second rigid tube.
Implementation 104: The method of Implementation 103, wherein the transfer kit further comprises one or more filters, wherein the one more filters are disposed in-line in at least one of the first rigid tube or the second rigid tube.
Implementation 105: The method of Implementation 96, further comprising inserting the radiation shield container into a radiation shielded pocket, the radiation shielded pocket covering one or more openings, wherein the one or more openings separate the external environment from the interior volume of the dispensing module; connecting the bulk solution container to a first tube disposed through the one or more openings; and transferring the radiopharmaceutical composition into the dispensing module through the first tube.
Implementation 106: The method of Implementation 105, the radiation shielded pocket further comprises a housing having an interior volume, where the interior volume of the housing is fluidly coupled to a lower opening and an upper opening; and a lid coupled to the housing and movable between an open position and a closed position, wherein the lid covers the upper opening when in a closed position.
Implementation 107: The method of Implementation 106, wherein inserting the radiation shielded container further comprises inserting the radiation shielded container through the lower opening into the interior volume of the housing.
Implementation 108: The method of Implementations 106 to 107, wherein connecting the bulk solution container further comprises moving the lid to the open position to access the bulk solution container and the first flexible tube through the upper opening; and connecting the outlet connector of the bulk solution container to the first tube.
Implementation 109: The method of any of Implementations 105 to 108, wherein the first tube is coupled to a transfer kit, the transfer kit disposed in an interior volume of the dispensing module, the transfer kit comprises a second tube having a first connector at one end of the tube and a second connector at an opposing end of the tube, wherein transferring the radiopharmaceutical composition further comprises coupling the outlet connector the bulk solution container to a first end of the first tube, the first end of the first tube being disposed in the external environment; coupling a second end of the first tube to the first connector of the transfer kit, the second end of the first tube being disposed in the interior volume of the dispensing module; moving the radiopharmaceutical composition from the bulk solution container portion through the first tube and through the transfer kit using a peristaltic pump, the peristaltic pump disposed in the interior volume of the dispensing module and coupled to the second tube of the transfer kit; and filtering the radiopharmaceutical composition between entry of the radiopharmaceutical composition into the first tube and the exit of the radiopharmaceutical composition from the transfer kit.
Implementation 110: The method of Implementation 109, wherein the transfer kit further comprises one or more filters, wherein the one more filters are disposed in-line in in the second tube of the transfer kit.
Implementation 111: A radiation shielded container, comprising a container portion having an interior volume and a first opening; a lid removably coupled to the container portion to cover the first opening and enclose the interior volume; and a bulk solution container disposed in the interior volume, the bulk solution container configured to contain a radiopharmaceutical composition.
Implementation 112: The radiation shielded container of Implementation 111, wherein the bulk solution container further comprises a bulk solution container portion for holding the radiopharmaceutical composition; and an outlet connector coupled to the bulk solution container portion.
Implementation 113: The radiation shielded container of Implementation 112, wherein the bulk solution container further comprises a backup outlet connector coupled to the bulk solution container portion.
Implementation 114: The radiation shielded container of Implementation 112, wherein the bulk solution container further comprises an inlet connector coupled to the bulk solution container portion.
Implementation 115: The radiation shielded container of Implementation 112, wherein the bulk solution container further comprises a vent connector coupled to the bulk solution container portion.
Implementation 116: The radiation shielded container of Implementation 112, wherein the container portion further comprises a second opening fluidly coupled to the interior volume of the container portion, the second opening for accessing the bulk solution container, wherein the outlet connector of the bulk solution container is configured to be accessible through the second opening.
Implementation 117: The radiation shielded container of Implementation 112, further comprising a secondary lid covering the first opening and disposed between the lid and the container portion, wherein the secondary lid comprises a third opening fluidly coupled to the interior volume of the container portion, wherein the outlet connector of the bulk solution container is configured to be accessible through the third opening.
Implementation 118: A modular manufacturing system, comprising a first unit to synthesize a radiopharmaceutical composition; a second unit to package the radiopharmaceutical composition into a radiopharmaceutical product; and a transport machine configured to transport the radiopharmaceutical composition from the first unit to the second unit, wherein at least a portion of the second unit has a higher grade manufacturing environment than at least a portion of the first unit.
Implementation 119: The modular manufacturing system of Implementation 118, wherein at least a portion of the first unit has a Grade D environment, and wherein at least a portion of the second unit has a Grade C environment.
Implementation 120: The modular manufacturing system of Implementation 118, wherein the first unit further comprising an isolator having an interior volume; and a synthesis module disposed in the interior volume, wherein the synthesis module is configured for producing a concentrated radiopharmaceutical composition.
Implementation 121: The modular manufacturing system of Implementation 120, wherein the interior volume of the isolator is a Grade D environment.
Implementation 122: The modular manufacturing system of Implementation 120, wherein the first unit further comprises a mother vial; a dilution container configured to hold a dilution solution; and a mixing container, wherein the mother vial is coupled to the synthesis module to receive the concentrated radiopharmaceutical composition and coupled to the dilution container to receive the dilution solution, wherein the mother vial is further coupled to the mixing container, wherein the mixing container is configured to receive a mixture of the concentrated radiopharmaceutical solution and the dilution solution from the mother vial and to homogenize the mixture into the radiopharmaceutical composition.
Implementation 123: The modular manufacturing system of Implementation 122, the isolator having a first opening and a second opening.
Implementation 124: The modular manufacturing system of Implementation 123, wherein the dilution container is disposed external to the interior volume of the isolator and coupled to the mother vial through the first opening.
Implementation 125: The modular manufacturing system of Implementation 123, further comprising a radiation shielded container having a bulk solution container disposed therein, wherein the bulk solution container configured to contain the radiopharmaceutical composition, wherein the transport machine having the radiation shielded container disposed thereon, and wherein the radiation shielded container and transport machine are disposed external to the interior volume of the isolator.
Implementation 126: The modular manufacturing system of Implementation 125, wherein the transport machine further comprises a mechanism for raising and lowering the radiation shielded container.
Implementation 127: The modular manufacturing system of Implementation 125, wherein the bulk solution container is coupled to the mixing container through the second opening.
Implementation 128: The modular manufacturing system of Implementation 127, wherein the radiation shielded container comprises a container portion having an interior volume and a first opening; and a lid removably coupled to the container portion to cover the first opening and enclose the interior volume, wherein the bulk solution container is disposed in the interior volume.
Implementation 129: The modular manufacturing system of Implementation 128, wherein the bulk solution container further comprises a bulk solution container portion for holding the radiopharmaceutical composition; and an outlet connector coupled to the bulk solution container portion.
Implementation 130: The modular manufacturing system of Implementation 129, wherein the bulk solution container further comprises a backup outlet connector coupled to the bulk solution container portion.
Implementation 131: The modular manufacturing system of Implementation 129, wherein the bulk solution container further comprises an inlet connector coupled to the bulk solution container portion, wherein the inlet connector is configured to be coupled to the mixing container via the second opening of the isolator.
Implementation 132: The modular manufacturing system of Implementation 129, wherein the bulk solution container further comprises a vent connector coupled to the bulk solution container portion.
Implementation 133: The modular manufacturing system of Implementation 129, wherein the container portion of the radiation shielded container further comprises a second opening fluidly coupled to the interior volume of the container portion, the second opening for accessing the bulk solution container, wherein the outlet connector of the bulk solution container is configured to be accessible through the second opening.
Implementation 134: The modular manufacturing system of Implementation 129, wherein the radiation shielded container further comprising a secondary lid covering the first opening and disposed between the lid and the container portion, wherein the secondary lid comprises a third opening fluidly coupled to the interior volume of the container portion, wherein the outlet connector of the bulk solution container is configured to be accessible through the third opening.
Implementation 135: The modular manufacturing system of Implementation 118, wherein the radiopharmaceutical product comprises the radiopharmaceutical composition disposed in a packaging material.
Implementation 136: The modular manufacturing system of Implementation 135, wherein the packaging material comprises a glass vial configured to have a cap fitted thereon, or a cartridge configured to be loaded into a syringe device.
Implementation 137: The modular manufacturing system of Implementation 118, further comprising a radiation shielded container having a bulk solution container disposed therein, wherein the bulk solution container configured to contain the radiopharmaceutical composition, and wherein the transport machine having the radiation shielded container disposed thereon.
Implementation 138: The modular manufacturing system of Implementation 118, wherein the second unit further comprises a dispensing module having a first interior volume.
Implementation 139: The modular manufacturing system of Implementation 138, wherein the first interior volume of the dispensing module is a Grade C environment.
Implementation 140: The modular manufacturing system of Implementation 138, wherein the dispensing module further comprises a radiation shielded pocket disposed externally from the first interior volume of the dispensing module, wherein the radiation shielded pocket is configured to interface with the radiation shielded container, wherein the radiation shielded pocket covers one or more openings in the dispensing module, wherein the one or more openings extend from the first interior volume of the dispensing module to the radiation shielded pocket, and wherein the one or more openings is configured to receive the radiopharmaceutical composition from the bulk solution container.
Implementation 141: The modular manufacturing system of Implementation 140, wherein the radiation shielded pocket further comprises a housing having a second interior volume, where the second interior volume of the housing is fluidly coupled to a lower opening and an upper opening, wherein the one or more openings of the dispensing module are disposed between the lower and upper openings; and a lid coupled to the housing and movable between an open position and a closed position, wherein the lid covers the upper opening when in a closed position, wherein the radiation shielded container is configured to enter the second interior volume of the housing through the lower opening.
Implementation 142: The modular manufacturing system of Implementation 141, wherein the radiation shielded container comprises a container portion having an interior volume and a first opening; and a lid removably coupled to the container portion to cover the first opening and enclose the interior volume, wherein the bulk solution container is disposed in the interior volume.
Implementation 143: The modular manufacturing system of Implementation 142, wherein the bulk solution container further comprises a bulk solution container portion for holding the radiopharmaceutical composition; and an outlet connector coupled to the bulk solution container portion.
Implementation 144: The modular manufacturing system of Implementation 143, wherein the bulk solution container further comprises a backup outlet connector coupled to the bulk solution container portion.
Implementation 145: The modular manufacturing system of Implementation 143, wherein the bulk solution container further comprises an inlet connector coupled to the bulk solution container portion.
Implementation 146: The modular manufacturing system of Implementation 143, wherein the bulk solution container further comprises a vent connector coupled to the bulk solution container portion.
Implementation 147: The modular manufacturing system of Implementation 143, wherein the container portion of the radiation shielded container further comprises a second opening fluidly coupled to the interior volume of the container portion, the second opening for accessing the bulk solution container, wherein the outlet connector of the bulk solution container is configured to be accessible through the second opening.
Implementation 148: The modular manufacturing system of Implementation 143, wherein the radiation shielded container further comprising a secondary lid covering the first opening and disposed between the lid and the container portion, wherein the secondary lid comprises a third opening fluidly coupled to the interior volume of the container portion, wherein the outlet connector of the bulk solution container is configured to be accessible through the third opening.
Implementation 149: The modular manufacturing system of Implementation 143, wherein each of the one or more openings of the dispensing module further comprise a first tube disposed therethrough from the first interior volume of the dispensing module to the second interior volume of the housing; a first connector coupled to one end of the first tube disposed in the second interior volume; a second connector coupled to an opposing end of the first tube disposed in the first interior volume; and a sealing device dispose in the opening such that the first interior volume is not fluidly coupled to the second interior volume.
Implementation 150: The modular manufacturing system of Implementation 149, further comprising a transfer kit for transferring the radiopharmaceutical composition from the bulk solution container into the first interior volume of the dispensing module via the one or more openings.
Implementation 151: The modular manufacturing system of Implementation 150, wherein the transfer kit further comprises a second tube having a first connector at one end of the second tube and a second connector at an opposing end of the second tube, wherein the first connector of the second tube is configured to be coupled to second connector of the first tube.
Implementation 152: The modular manufacturing system of Implementation 151, wherein the transfer kit further comprises one or more filters for decontaminating the radiopharmaceutical composition upon exit from the bulk solution container, wherein the one more filters are disposed in-line in the second tube.
Implementation 153: The modular manufacturing system of Implementation 152, further comprising an auxiliary line coupled to a vent connector disposed on the one or more filters.
Implementation 154: The modular manufacturing system of Implementation 151, further comprising a peristaltic pump coupled to the second tube of the transfer kit to facilitate movement of the radiopharmaceutical composition therethrough.
Implementation 155: The modular manufacturing system of Implementation 149, wherein the first connector of the first tube is configured to be coupled to the outlet connector of the bulk solution container.
Implementation 156: The modular manufacturing system of Implementation 128, wherein the bulk solution container further comprises a bulk solution container portion for holding the radiopharmaceutical composition; an outlet connector coupled to the bulk solution container portion; and an inlet connector coupled to the bulk solution container portion, wherein the inlet connector is configured to be coupled to the mixing container via the second opening of the isolator.
Implementation 157: The modular manufacturing system of Implementation 156, wherein the bulk solution container further comprises a backup outlet connector coupled to the bulk solution container portion; and a vent connector coupled to the bulk solution container portion.
Implementation 158: The modular manufacturing system of Implementation 156, wherein the radiation shielded container further comprising a secondary lid covering the first opening and disposed between the lid and the container portion, wherein the secondary lid comprises a third opening fluidly coupled to the interior volume of the container portion, wherein the outlet connector of the bulk solution container is configured to be accessible through the third opening.
Implementation 159: The modular manufacturing system of Implementation 156, wherein the second unit further comprises a dispensing module having a first interior volume.
Implementation 160: The modular manufacturing system of Implementation 159, wherein the first interior volume of the dispensing module is a Grade C environment.
Implementation 161: The modular manufacturing system of Implementation 159, wherein the dispensing module further comprises a radiation shielded pocket disposed externally from the first interior volume of the dispensing module, wherein the radiation shielded pocket is configured to interface with the radiation shielded container, wherein the radiation shielded pocket covers one or more openings in the dispensing module, wherein the one or more openings extend from the first interior volume of the dispensing module to the radiation shielded pocket, and wherein the one or more openings is configured to receive the radiopharmaceutical composition from the bulk solution container.
Implementation 162: The modular manufacturing system of Implementation 161, wherein the radiation shielded pocket further comprises a housing having a second interior volume, where the second interior volume of the housing is fluidly coupled to a lower opening and an upper opening, wherein the one or more openings of the dispensing module are disposed between the lower and upper openings; and a lid coupled to the housing and movable between an open position and a closed position, wherein the lid covers the upper opening when in a closed position, wherein the radiation shielded container is configured to enter the second interior volume of the housing through the lower opening.
Implementation 163: The modular manufacturing system of Implementation 161, wherein each of the one or more openings of the dispensing module further comprise a first tube disposed therethrough from the first interior volume of the dispensing module to the second interior volume of the housing; a first connector coupled to one end of the first tube disposed in the second interior volume; a second connector coupled to an opposing end of the first tube disposed in the first interior volume; and
a sealing device dispose in the opening such that the first interior volume is not fluidly coupled to the second interior volume.
Implementation 164: The modular manufacturing system of Implementation 163, further comprising a transfer kit for transferring the radiopharmaceutical composition from the bulk solution container into the first interior volume of the dispensing module via the one or more openings.
Implementation 165: The modular manufacturing system of Implementation 164, wherein the transfer kit further comprises a second tube having a first connector at one end of the second tube and a second connector at an opposing end of the second tube, wherein the first connector of the second tube is configured to be coupled to second connector of the first tube.
Implementation 166: The modular manufacturing system of Implementation 165, wherein the transfer kit further comprises one or more filters for decontaminating the radiopharmaceutical composition upon exit from the bulk solution container, wherein the one more filters are disposed in-line in the second tube.
Implementation 167: The modular manufacturing system of Implementation 165, further comprising an auxiliary line coupled to a vent connector disposed on the one or more filters.
Implementation 168: The modular manufacturing system of Implementation 165, further comprising a peristaltic pump coupled to the second tube of the transfer kit to facilitate movement of the radiopharmaceutical composition therethrough.
Implementation 169: The modular manufacturing system of Implementation 163, wherein the first connector of the first tube is configured to be coupled to the outlet connector of the bulk solution container. Implementation 170: A method of manufacturing, comprising synthesizing a radiopharmaceutical composition in a first unit; packaging the radiopharmaceutical composition into a radiopharmaceutical product in a second unit; and transporting the radiopharmaceutical composition synthesized in the first unit to the second unit for packaging, wherein at least a portion of the second unit has a higher grade manufacturing environment than at least a portion of the first unit.
Implementation 171: The method of Implementation 170, wherein at least a portion of the first unit has a Grade D environment, and wherein at least a portion of the second unit has a Grade C environment.
Implementation 172: The method of Implementation 170, further comprising loading the radiopharmaceutical composition synthesized in the first unit into a bulk solution container.
Implementation 1732: The method of Implementation 172, wherein the bulk solution container further comprises a bulk solution container portion for holding the radiopharmaceutical composition; an outlet connector coupled to the bulk solution container portion; and an inlet connector coupled to the bulk solution container portion.
Implementation 174: The method of Implementation 173, wherein the bulk solution container further comprises a backup outlet connector coupled to the bulk solution container portion.
Implementation 175: The method of Implementation 173, wherein the bulk solution container further comprises a vent connector coupled to the bulk solution container portion.
Implementation 176: The method of Implementation 173, wherein loading the radiopharmaceutical composition further comprises loading the radiopharmaceutical composition through the inlet connector into the bulk solution container portion.
Implementation 177: The method of Implementation 176, wherein the bulk solution container is disposed in a radiation shielded container, the radiation shielded container comprising a container portion having an interior volume and a first opening; and a lid removably coupled to the container portion to cover the first opening and enclose the interior volume, wherein the bulk solution container is disposed in the interior volume of the container portion.
Implementation 178: The method of Implementation 177, wherein the container portion further comprises a second opening fluidly coupled to the interior volume of the container portion, the second opening for accessing the bulk solution container disposed in the interior volume of the container portion.
Implementation 179: The method of Implementation 177, wherein the radiation shielded container further comprises a secondary lid covering the first opening and disposed between the lid and the container portion, wherein the secondary lid comprises a third opening fluidly coupled to the interior volume of the container portion.
Implementation 180: The method of Implementation 177, wherein the first unit further comprising an isolator having an interior volume; and a synthesis module disposed in the interior volume of the isolator, wherein the synthesis module is configured for producing a concentrated radiopharmaceutical composition.
Implementation 181: The method of Implementation 180, wherein the interior volume of the isolator is a Grade D environment.
Implementation 182: The method of Implementation 180, wherein the first unit further comprises a mother vial; a dilution container configured to hold a dilution solution; and a mixing container, wherein the mother vial is coupled to the synthesis module to receive the concentrated radiopharmaceutical composition and coupled to the dilution container to receive the dilution solution, wherein the mother vial is further coupled to the mixing container, wherein the mixing container is configured to receive a mixture of the concentrated radiopharmaceutical solution and the dilution solution from the mother vial and to homogenize the mixture into the radiopharmaceutical composition.
Implementation 183: The method of Implementation 182, the isolator having a first opening and a second opening.
Implementation 184: The method of Implementation 183, wherein the dilution container is disposed external to the isolator and coupled to the mother vial through the first opening.
Implementation 185: The method of Implementation 183, wherein the radiation shielded container having the bulk solution container disposed therein is disposed on a transportation machine and external to the isolator.
Implementation 186: The method of Implementation 185, wherein loading the radiopharmaceutical composition further comprises coupling the inlet connector of the bulk solution container to a corresponding connector of a transfer kit extending through the first opening in the isolator, wherein the transfer kit is coupled to the mixing container; and loading the radiopharmaceutical composition from the mixing container into the bulk solution container portion of the bulk solution container.
Implementation 187: The method of 186, further comprising moving the transport machine having the radiation shielded container with the bulk solution container and radiopharmaceutical composition therein to the second unit.
Implementation 188: The method of Implementation 170, further comprising unloading the radiopharmaceutical composition synthesized in the first unit from a bulk solution container into the second unit.
Implementation 189: The method of Implementation 188, wherein the bulk solution container further comprises a bulk solution container portion for holding the radiopharmaceutical composition; an outlet connector coupled to the bulk solution container portion; and an inlet connector coupled to the bulk solution container portion.
Implementation 190: The method of Implementation 189, wherein the bulk solution container further comprises a backup outlet connector coupled to the bulk solution container portion.
Implementation 191: The method of Implementation 189, wherein the bulk solution container further comprises a vent connector coupled to the bulk solution container portion.
Implementation 192: The method of Implementation 189, wherein the bulk solution container is disposed in a radiation shielded container, the radiation shielded container comprising a container portion having an interior volume and a first opening; and a lid removably coupled to the container portion to cover the first opening and enclose the interior volume, wherein the bulk solution container is disposed in the interior volume of the container portion.
Implementation 193: The method of Implementation 192, wherein the container portion further comprises a second opening fluidly coupled to the interior volume of the container portion, the second opening for accessing the bulk solution container disposed in the interior volume of the container portion.
Implementation 194: The method of Implementation 192, wherein the radiation shielded container further comprises a secondary lid covering the first opening and disposed between the lid and the container portion, wherein the secondary lid comprises a third opening fluidly coupled to the interior volume of the container portion.
Implementation 195: The method of Implementation 192, wherein the second unit further comprising a dispensing module having a first interior volume.
Implementation 196: The method of Implementation 195, wherein the first interior volume of the dispensing module is a Grade C environment.
Implementation 197: The method of Implementation 195, wherein the dispensing module further comprises a radiation shielded pocket disposed externally from the first interior volume of the dispensing module, wherein the radiation shielded pocket is configured to interface with the radiation shieled container, wherein the radiation shielded pocket covering one or more openings in the dispensing module, where the one or more openings extend from the first interior volume of the dispensing module to the radiation shielded pocket, and wherein the one or more openings is configured to receive the radiopharmaceutical composition from the bulk solution container.
Implementation 198: The method of Implementation 197, wherein the radiation shielded pocket further comprises a housing having a second interior volume, where the second interior volume of the housing is fluidly coupled to a lower opening and an upper opening, wherein the one or more openings of the dispensing module are disposed between the lower and upper openings; and a lid coupled to the housing and movable between an open position and a closed position, wherein the lid covers the upper opening when in a closed position, wherein the radiation shielded container is configured to enter the second interior volume of the housing through the lower opening.
Implementation 199: The method of Implementation 198, wherein each of the one or more openings of the dispensing module further comprise a first tube disposed therethrough from the first interior volume of the dispensing module to the second interior volume of the housing; a first connector coupled to one end of the first tube disposed in the second interior volume; a second connector coupled to an opposing end of the first tube disposed in the first interior volume; and a sealing device dispose in the opening such that the first interior volume is not fluidly coupled to the second interior volume.
Implementation 200: The method of Implementation 199, further comprising inserting the radiation shield container into a radiation shielded pocket; connecting the bulk solution container to the first tube; and transferring the radiopharmaceutical composition into the dispensing module through the first tube.
Implementation 201: The method of Implementation 200, wherein inserting the radiation shielded container further comprises inserting the radiation shielded container through the lower opening into the interior volume of the housing.
Implementation 202: The method of Implementations 200, wherein connecting the bulk solution container further comprises moving the lid to the open position to access the bulk solution container through the upper opening; and connecting the outlet connector of the bulk solution container to the first tube.
Implementation 203: The method of Implementation 202, where the dispensing module further comprising a transfer kit for transferring the radiopharmaceutical composition from the bulk solution container into the first interior volume of the dispensing module via the one or more openings, wherein the transfer kit further comprises a second tube having a first connector at one end of the second tube and a second connector at an opposing end of the second tube, wherein the first connector of the second tube is configured to be coupled to second connector of the first tube.
Implementation 204: The method of Implementation 203, wherein the transfer kit further comprises one or more filters for decontaminating the radiopharmaceutical composition upon exit from the bulk solution container, wherein the one more filters are disposed in-line in the second tube.
Implementation 205: The method of Implementation 204, further comprising a peristaltic pump coupled to the second tube of the transfer kit to facilitate movement of the radiopharmaceutical composition therethrough.
Implementation 206: The method of Implementation 205, wherein transferring the radiopharmaceutical composition further comprises coupling the outlet connector the bulk solution container to a first connector of the first tube; moving the radiopharmaceutical composition from the bulk solution container portion through the first tube and through the transfer kit using the peristaltic pump; and filtering the radiopharmaceutical composition between entry of the radiopharmaceutical composition into the first tube and the exit of the radiopharmaceutical composition from the transfer kit.
Implementation 207: The method of Implementation 206, further comprising loading the radiopharmaceutical composition, after exit from the transfer kit, into packing material.
Implementation 208: The method of Implementation 207, wherein the packing material comprises a glass vial configured to have a cap fitted thereon, or a cartridge configured to be loaded into a syringe device.
Implementation 209: The method of Implementation 170, wherein the radiopharmaceutical product comprises the radiopharmaceutical composition disposed in a packaging material.
Implementation 210: The method of Implementation 208, wherein the packaging material comprises a glass vial configured to have a cap fitted thereon, or a cartridge configured to be loaded into a syringe device.

It should be appreciated that all combinations of the foregoing concepts and additional concepts discussed in greater detail below (provided such concepts are not mutually inconsistent) are contemplated as being part of the inventive subject matter disclosed herein and may be employed to provide the technical benefits described herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows, in one example, a schematic of one example set up of a radiopharmaceutical manufacturing process.

FIG. 2 shows, in one example, a schematic of one example set up of a modular radiopharmaceutical manufacturing process.

FIG. 3 shows, in one example, a schematic of workflow of the operations taking place in a synthesis module in one example set up described herein.

FIG. 4 shows, in one example, a schematic of the workflow illustrating the processes in one example set up described herein.

FIG. 5 shows, in one example, a schematic of the operations taking place in an example dispensing module in one example set up described herein.

FIG. 6 shows, in one example, a schematic of the workflow illustrating the processes into and out of a synthesis module in one example set up described herein.

FIG. 7 shows, in one example, a schematic of a modular radiopharmaceutical manufacturing arrangement employing trains between modules.

FIG. 8 shows, in one example, a schematic of an automated synthesis and formulation module that may be incorporated into the arrangement of FIG. 7.

FIG. 9 shows, in one example, a schematic of an automated dispensing module that may be incorporated into the arrangement of FIG. 7.

FIG. 10 shows, in one example, a schematic of an automated inspection, labelling, and assembly module that may be incorporated into the arrangement of FIG. 7.

FIG. 11 shows, in one example, a schematic of an arrangement where a set of remote computing devices are coupled with a radiopharmaceutical manufacturing system.

FIG. 12 shows, in one example, a flow chart of a process that may be carried out via the arrangement of FIG. 11.

FIG. 13 shows, in one example, a schematic of a data flow that may be carried out in an implementation of the process of FIG. 12.

FIG. 14A shows, in one example, a schematic view of a radiation shielded container having a bulk solution container disposed therein.

FIG. 14B shows, in one example, a schematic view of a radiation shielded container having a bulk solution container disposed therein.

FIG. 15A shows, in one example, a schematic view of a first unit and a transport machine.

FIG. 15B shows, in one example, a schematic view of a first unit and a transport machine.

FIG. 15C shows, in one example, a schematic view of a first unit and a transport machine.

FIG. 16A shows, in one example, a schematic view of a second unit and a transport machine.

FIG. 16B to 16D show, in one example, a schematic view of a second unit and a transport machine.

FIG. 16E to 16F show, in one example, a schematic view of a second unit and a transport machine.

FIG. 17 shows, in one example, a transfer kit for transferring a radiopharmaceutical composition from a bulk solution container.

### DETAILED DESCRIPTION

Following below are more detailed descriptions of various concepts related to, and implementations of, inventive systems and methods related to manufacturing of radiopharmaceutical products. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### RLT

The systems and methods described herein may be employed to provide the benefits of overcoming challenges encountered during manufacturing of radiopharmaceutical products, e.g., manufacturing of radioligand therapeutic (“RLT”) products.

### Radiopharmaceutical Drug Product

In certain examples provided herein, the term radiopharmaceutical drug product may refer to any drug product that comprises a radioactive material, e.g., a radioactive nuclide, radionuclide or radioisotope, e.g., F-18, Cu-64, Cu-67, Ga-68, Y-90, Tc-99m, I-131, In-111, Tb-161, Er-169, Lu-177, Pb-212, Bi-212, Ra-223, or Ac-225. The radionuclide may be present in such drug product in the form of a salt, e.g., as aqueous solution (e.g., Radium chloride solution, Xofigo; Lutetium chloride solution, Lutathera), or covalently bound to an organic molecule, e.g., 18F-DCFPyL, Pylarify), or complexed in a chelator, whereby said chelator might be conjugated (e.g., via a linker) to a target-binding moiety, e.g., a radioligand, e.g., 177Lu-DOTATATE, Lutathera, or 177Lu-PSMA-617, Pluvicto.

### Radioligand Drug Product

In certain examples provided herein, the term radioligand drug product may refer to a drug product comprising a radioligand for therapeutic use (e.g., a radioligand therapeutic (RLT) agent) or for imaging use (i.e., a radio ligand imaging (RLI) agent). The RLT agent may comprise at least two components: (1) a radionuclide component; and (2) a ligand component; wherein said radionuclide component (1) comprises: (a) at least one radionuclide, in some examples selected from the group consisting of alpha particle-emitting radionuclide, beta-minus electron-emitting radionuclide and Auger electron-emitting radionuclide (in one example a beta-minus electronemitting radionuclide); and wherein said ligand component (2) comprises: (b) at least one target binding moiety (e.g., a PSMA-binding moiety, or an somatostatin receptor targeting moiety); (c) optionally at least one chelator for chelating the radionuclide or a salt comprising the radionuclide, or a prosthetic group residue from a radiohalogenation reaction; (d) optionally at least one linker connecting the PSMA-binding moiety (b) with the chelator or prosthetic group component (c), in some examples the linker is a chemical moiety or a covalent bond; (e) optionally at least one additional moiety that alters the systemic circulation time, tumor uptake, and/or biodistribution of the RLT agent, in some examples the altering moiety comprises oxyethylene units, e.g., an oligo- or polyoxyethylene —(—CH2-CH2-O-)n- with n=2-100, or is an albumin-binding moiety (e.g., Evans blue, 4-(p-iodophenyl)butyric acid, 4-(p-methylphenyl)butyric acid, ibuprofen).

The radionuclide may be selected from the group consisting of Lu-177, Tb-161, I-131, Tc-99m, Y-90, Sc-47, Cu-67, Re-188, Pb-212, Bi-213, Ac-255, and Th-227. In one example, it is selected from the group consisting of Lu-177, Ac-255, and Tb-161.

The radionuclide may be a beta-minus electron emitting radionuclide with an half-life of from about 2 to about 10 days, in some examples from about 5 to about 10 days—e.g., from about 6 to about 8 days, such as about 6, and about 7 days; and a beta-minus electron maximum energy of from about 0.3 to about 1.0 MeV—e.g., from about 0.5 to about 0.8 MeV, such as 0.5, 0.6, 0.7, or 0.8 MeV; in one example, the energy is about 0.5 or about 0.6 MeV.

The radionuclide may be a beta-minus electron emitting radionuclide with an absorbed electron energy fraction per decay of from 100 to 300 keV/decay, from 120 to 250 keV/decay, from about 150 keV/decay (e.g., for Lu-177: 147 keV) to about 200 keV/decay (e.g., for Tb-161: 196 keV/decay). The ligand may be selected from the group consisting of PSMA-617, PSMA 1&T, PSMA-R2, MIP-1095, MIP-1545, MIP-1555, MIP-1557, MIP-1558, CTT1403, FC705, BAY-2315497, TLX592, PSMA-TCC, rhPSMA, rhPSMA-7, rhPSMA-7.3, rhPSMA-10.1, Ludotadipep, PNT2001, PNT2002, PSMA-7 l&T, EB-PSMA-617, PSMA-ALB-02, PSMA-ALB-053, PSMA-ALB-056, P16-093, PSMA-93, and RPS-074 (or any albumin-binder-modified versions thereof, e.g., Evans blue (EB)-PSMA-617). In one example, it is selected from the group consisting of PSMA-617, PSMA 1&T, and PSMA-R2.

The PSMA-binding moiety may comprise at least two amino acids connected via an urea or phosphoramide group—e.g., glutamate-urea-lysine (GUL), or an antibody or fragment thereof, e.g., TLX591, J591, rosopatamab, IAB2M, GCP-05, 1 H8H5, SP29, or FOLHI.

The radioligand therapeutic agent may be selected from the group consisting of [177Lu]Lu-PSMA-617 (lutetium (177Lu) vipivotide tetraxetan), [177Lu]Lu-EB-PSMA-617 (Evans Blue modified [177Lu]Lu-PSMA-617), and [177Lu]Lu-PSMA 1&T (lutetium (177Lu) zadavotide guraxetan), [161Tb]Tb-PSMA-617 (terbium (161Tb) vipivotide tetraxetan), [161Tb]Tb-EB-PSMA-617 (Evans Blue modified [161Tb]Tb-PSMA-617), and [161Tb]Tb-PSMA l&T (terbium (161Tb) zadavotide guraxetan), in some examples [177Lu]Lu-PSMA-617 (lutetium (177Lu) vipivotide tetraxetan) or [161Tb]Tb-PSMA-617 (terbium (161Tb) vipivotide tetraxetan), in one example [177Lu]Lu-PSMA-617 (lutetium (177Lu) vipivotide tetraxetan).

The PSMA-binding moiety may be glutamate-urea-lysine (GUL) and the linker may comprise the residues of (2-naphthyl)-L-alanine and trans-4-aminomethyl-cyclohexanecarboxylic acid or the linker comprises the residues of an optionally substituted phenyl-alanine and/or optionally substituted tyrosine. In one example, it is a phenyl-alanine and a substituted tyrosine. In one example, it is a phenyl-alanine and an iodo-substituted tyrosine. In one example, it is a D-phenyl-alanine and an iodo-substituted D-tyrosine.

The ligand may be selected from the group consisting of DOTA-OC: [DOTAO, D-Phe1]octreotide, DOTA-TOC: [DOTAO, D-Phe1,Tyr3]octreotide, edotreotide (INN), DOTA-NOC: [DOTAO, D-Phe1,1-Nal3]octreotide, DOTA-TATE: [DOTAO, D-Phe1,Tyr3]octreotate, DOTA-Tyr3-Octreotate, DOTA-d-Phe-Cys-Tyr-d-Trp-Lys-Thr-Cys-Thr (cyclo 2,7), oxodotreotide (INN), DOTA-LAN: [DOTAO, D-p-Nal1]lanreotide, DOTA-VAP: [DOTAO, D-Phe1,Tyr3]vapreotide, Satoreotide trizoxetan, and Satoreotide tetraxetan. The radioligand imaging agent may comprise: (1) a radionuclide component; and (2) a ligand component; wherein said radionuclide component (1) comprises: (a) at least one positron-emitting radionuclide; and wherein said ligand component (2) comprises: (b) at least one target-binding moiety (e.g., a PSMA-binding moiety, or an somatostatin receptor binding peptide, e.g., oxodotreotide or edotreotide); (c) optionally at least one chelator for chelating the radionuclide or a salt comprising the radionuclide, or a prosthetic group residue from a radiohalogenation reaction; (d) optionally at least one linker connecting the PSMA binding moiety (b) with the chelator/prosthetic group component (c), in some examples the linker is a chemical moiety or a covalent bond. The radionuclide for the RLI agent may be selected from the group consisting of F-18, Ga-67, Ga-68, and Cu-64. The ligand for the RLI agent may be selected from the group consisting of PSMA-11 (gozetotide), DCPyL (if labeled with 18F available as PYLARIFY, INN: piflufolastat F-18, also referred to shortly as PyL), MIP-1404, rhPSMA 07, PSMA-1007, THP-PSMA, iPSMA, Pie-093, PSMA-93, rhPSMA, rhPSMA-7, rhPSMA-7.3, PSMA-7, and PSMA 1&T. The ligand may be selected from the group consisting of DOTA-OC: [DOTAO, D-Phe1]octreotide, DOTA-TOC: [DOTAO,D-Phe1,Tyr3]octreotide, edotreotide (INN), DOTA-NOC: [DOTAO, D-Phe1,1-Nal3]octreotide, DOTA-TATE: [DOTAO, D-Phe1,Tyr3]octreotate, DOTA-Tyr3-Octreotate, DOTA-d-Phe-Cys-Tyr-d-Trp-Lys-Thr-Cys-Thr (cyclo 2,7), oxodotreotide (INN), DOTA-LAN: [DOTAO, D-p-Nal1]lanreotide, DOTA-VAP: [DOTAO, D-Phe1,Tyr3]vapreotide, Satoreotide trizoxetan, and Satoreotide tetraxetan.

### Synthesizing

In certain examples provided herein, the term “synthesizing” may refer to radiolabeling a target-binding ligand, e.g., by complexing a radionuclide into the chelator of the ligand. The radiolabeling may involve heating a solution of the radionuclide and the ligand for a certain time (e.g., about 5-15 min) and at a certain temperature (e.g., about 95° C.). The solution may contain a buffer for ensuring the pH is optimal for the complex formation (e.g., an acetate buffer for a pH of about 4-6), a stabilizer against radiolytic degradation (autoradiolysis), e.g., gentisic acid or ascorbic acid, or salts thereof. The result of this radiolabeling may be referred to as mother solution or concentrated mother solution.

The synthesizing process may be performed in a synthesis module. The synthesis module may be a cassette-based synthesis module. In one example, the cassette-system manufacturing synthesizer unit as provided e.g., by Trasis, in some examples MiniAIO as described e.g., in WO 2020/089379 A1 and WO 2024/1231848 A1. In one example, two double synthesis modules by Trasis are used.

In some examples, the synthesis process may include a compounding process. In certain examples provided herein, the term “compounding” may refer to diluting the concentrated mother solution, e.g., by adding water or an aqueous solution, which in some examples may further comprise a sequestering agent (e.g., DTPA), a stabilizer against radiolytic degradation (autoradiolysis), e.g., gentisic acid or ascorbic acids, or salts thereof, and in some examples may further comprise an isotonic agent, e.g., NaCl, to adjust the tonicity of the resulting solution; and in some examples may further comprising a pH adjusting agent, e.g., NaOH or HCl to adjust the pH to bring the pH closer to physiological pH. One objective of this dilution process is to adjust the radioactivity concentration to a target concentration that is then suitable for the filling process to produce the patient dose units, e.g., about 1000 MBq/mL (27 mCi/mL).

The compounding process may comprise the sub-processes of. (i) filling the synthesized solution(s) into a container, e.g., a (bulk) vial or bottle to obtain a (concentrated) mother solution; (ii) optionally, measuring the radioactivity of said (concentrated) mother solution; (iii) optionally, transferring said (concentrated) mother solution into another container, in some examples a flexible plastic bag, (iv) adding a dilution solution in an amount to obtain the drug product solution in the desired radioactive concentration; and (v) mixing the solution as result of process (iv), if applicable in some examples, by moving the flexible plastic bag appropriately, to obtain a homogeneous diluted drug product solution for filling.

### Shield

In certain examples provided herein, the term “shielded unit” may refer to a manufacturing unit that is shielded by a material suitable to reduce and/or prevent ionizing radiation (e.g., alpha-particle, beta-minus electrons, gamma-rays) from leaving said unit. Therefore, within the shielded units, radioactive material can be safely handled.

### Cold Unit

In certain examples provided herein, the term “cold unit” may refer to those manufacturing units that are not shielded against ionizing radiation. Therefore, in those cold units, no radioactive material is handled.

### Primary Packaging Material

In certain examples provided herein, the term “primary packaging material” may refer to those containers that are in direct contact with the drug product, e.g., with the radioactive solution. Such primary packaging material may be a glass vial with a rubber stopper and a cap; or a glass vial with an all-plastic push-fit cap, wherein the rubber stopper is preassembled in the cap, for example, a RayDyLyo cap by Araymond; or a cartridge, e.g., a glass cylinder that is closed by rubber pieces, wherein at least one rubber piece is movable, said cartridge may be then used by loading into syringe devices; or a prefilled syringe, such as a prefilled glass syringe. Such primary packaging material may be provided in tubs that are sealed, e.g., by foils, such as those commercialized under the name Tyvek by DuPont (e.g., made out of high-density polyethylene (HDPE)). Such sealed tubs containing the primary packaging material may be then wrapped in a further bag.

### Inspecting

In certain examples provided herein, the term “inspecting” may refer to processes that control the quality and integrity of the drug product. Those inspecting or inspection processes may be in-process controls or final product controls. Those inspection processes may be container closure integrity tests (CCIT), radioactive dose measurement/calibration, visual inspection, head space analysis, or any other product quality controls.

### Cleanliness Zoning Designation:

Pharmaceutical manufacturing environments are classified into standardized grades to control the potential for contamination and thereby ensure product safety, purity, and quality. Accordingly, the different manufacturing units described herein may have different (manufacturing) environments characterized by different grades. For example, the environment may be designated as Grade A, B, C, or D. Grade A environments provide the highest level of cleanliness and contamination control. Grade B environments provide a lower level of cleanliness and contamination control yet provide a controlled environment around critical Grade A operations. A Grade B environment may serve as a background for a Grade A environment, supporting aseptic manufacturing processes. Grade C environments are characterized by intermediate cleanliness and contamination control and ISO 7 and ISO 8 classifications as shown below. Grade D environments are characterized by the least stringent controls. Unlike the higher grades, Grade D does not meet any of the formal pre-determined grades of cleanliness during operation, but instead is maintained at an appropriate, manufacturer-defined level of cleanliness. In some embodiments, the different Grades described herein follow the industry standards as described below. Clean room and clean air devices are classified in accordance with EN ISO 14644-1. The maximum permitted airborne particle concentration for each grade is given in the following table.

| | Maximum permitted number of particles per m3 equal | | | |
|---|---|---|---|---|
| | to or greater than the tabulated size | | | |
| | At Rest | | In Operation | |
| Grade | 0.5 μm | 5.0 μm | 0.5 μm | 5.0 μm |
| A | 3 520 | 20 | 3 520 | 20 |
| B | 3 520 | 29 | 352 000 | 2 900 |
| C | 352 000 | 2 900 | 3 520 000 | 29 000 |
| D | 3 520 000 | 29 000 | Not Defined | Not Defined |

The approximate airborne particle classification per ISO 14644-1 standards is listed in the table below.

| Approximate airborne particle classification | |
|---|---|
| per ISO 14644-1 standards | |
| At Rest | In Operation |

| Grade | 0.5 μm | 5.0 μm | 0.5 μm | 5.0 μm |
|---|---|---|---|---|
| A | ISO 5 | ISO 5 | ISO 5 | ISO 5 |
| B | ISO 5 | ISO 5 | ISO 7 | ISO 7 |
| C | ISO 7 | ISO 7 | ISO 8 | ISO 8 |
| D | ISO 8 | ISO 8 | Not Defined | Not Defined |

The Grade “Controlled, Non-Classified” (CNC) refers to a cGMP manufacturing area designed to produce a consistent and controlled environment, but not necessarily monitored to a given environmental classification. Non-classified areas may have uncontrolled airborne particle amounts, but temperature and humidity are still maintained. Non-classified area may be a non-process area or area in which the product does not come in direct contact with air.

Certain sanitizing processes and equipment may be employed to achieve the targeted level of cleanness, and thus the Grade for the room. The sanitizing process may for example include sterilizing the composition that is being processed.

In some embodiments, the modular manufacturing system disclosed herein advantageously leverages lower grade environments. Maintaining pharmaceutical manufacturing environments at higher cleanliness grades imposes substantial operational and cost burdens that escalate non-linearly with each step of improvement. The HVAC system, which represents a substantial proportion of total cleanroom construction cost, must deliver exponentially greater air change rates at higher grades—an ISO 5 (Grade A) cleanroom requires 300-480 air changes per hour compared to only 20 air changes per hour for an ISO 8 (Grade D) environment—resulting in dramatically higher capital expenditure and ongoing energy consumption. Beyond construction, operational expenses are dominated by HVAC energy costs representing 50-70% of operating expenses, with higher-grade areas additionally burdened by requalification requirements twice annually versus annually for lower grades, along with more frequent filter replacement and preventive maintenance. Furthermore, higher grades necessitate more complex facility infrastructure, including multiple airlocks and additional partitions, that lower-grade areas do not require. Consequently, configuring manufacturing devices and processes to operate across both lower- and higher-cleanliness support grades-rather than confining all operations to higher grande environments-enables manufacturers to reduce capital expenditure, substantially lower ongoing operational energy costs, extend requalification intervals, and achieve significant cost savings while maintaining full regulatory compliance and product quality.

### Modular RLT Manufacturing Process

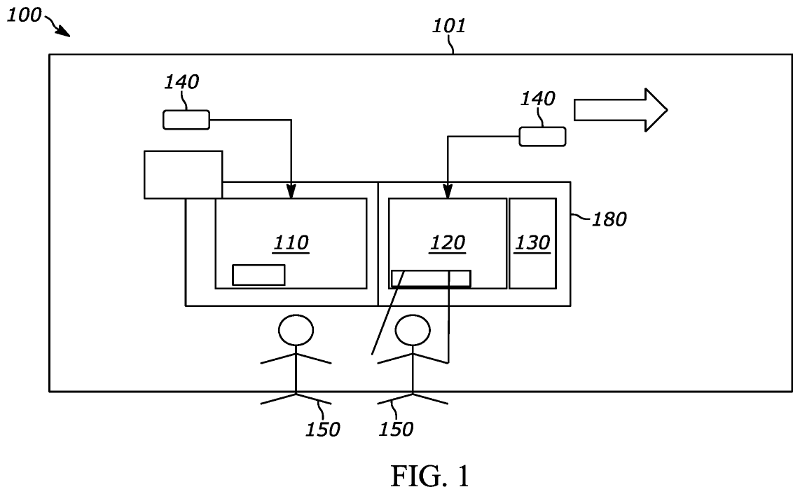

FIG. 1 illustrates a schematic of an example preexisting arrangement 100 of an RLT manufacturing process. As shown in the figure, the different modules are contained in one single unit 101, which may be a chamber. It is noted that the unit 101 may have any suitable configuration, such as a chamber (e.g., clean room), and even though in some instances the term “chamber” is used herein only to illustrate the concept of a “unit”, the term “chamber” is not meant to be limiting, and instead it refers to the “unit” as described herein. The chamber of the unit 101 may be a clean room of any suitable size, and the room may have a Grade C environment. Any suitable techniques may be employed to maintain the Grade for the environment of the chamber.

In the present example, the unit 101 contains a synthesis module 110, a dispensing module 120, and a sanitizing module 130. The synthesis module 110 may have any suitable hardware and equipment that may be employed to perform any of the synthesis processes described herein (e.g., radiolabeling reactions). The dispensing module 120 may have any suitable hardware or equipment that may be employed to perform any of the dispensing and packaging processes described herein. The sanitizing module 130 may have any suitable hardware or equipment that may be employed to perform any of the sanitizing processes described herein.

In the present example, the synthesis module 110, dispensing module 120, and the sanitizing module 130 is enclosed by a shield 180. The shield 180 may be any of the shields described herein. The synthesis module 110 may further comprise one or more of a reactor, and reservoirs/receptacles for radionuclide source, ligand source, and reaction buffer. The dispensing module 120 may further comprise one or more of a filling robot, and reservoirs/receptacles for drug substance, chelator, dilution solution, water for injection, and drug product. Additional components, such as pumps, valves, manifolds, tubes, etc., may be present. The production of radiopharmaceuticals occurs in the synthesis module 110 and is carried out every time with consumables, such as a new cassette 140, new set of reagents, and new tubing. All consumables are sterile and single use. At least one operator 150 is present.

In the example arrangement 100, as shown in FIG. 1, it is noted that the synthesis module 110, dispensing module 120, and the sanitizing module 130 are physically contiguous, all contained in one unit 101, and the unit 101 is operated in a Grade C environment. Also, the dispensing module 120 is operated manually by the at least one operator 150. Because of the manual process involved in this example setup, in some examples multiple operators 150 are involved.

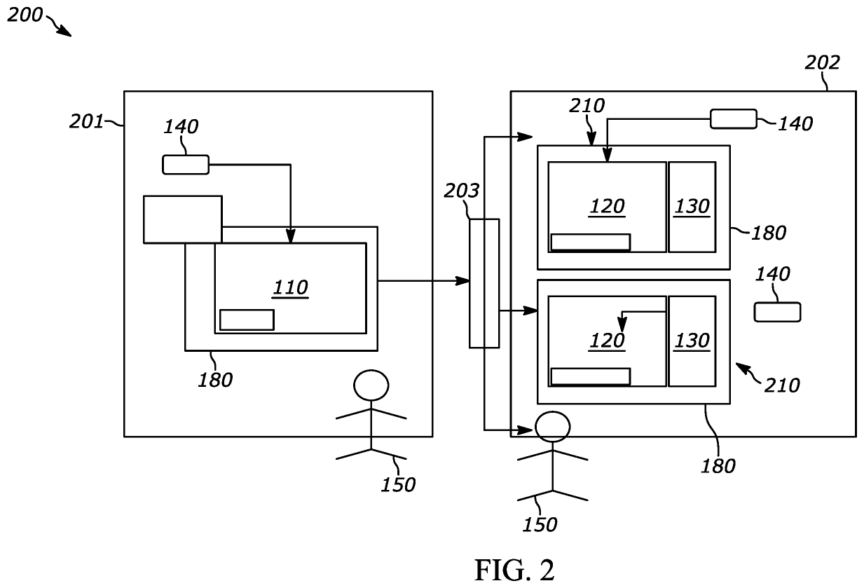

FIG. 2 illustrates a schematic of an example arrangement 200 of a modular RLT manufacturing process as described herein. In this modular setup, the different modules 110, 120, 130 may be the same as those shown in FIG. 1. However, in this modular setup, the synthesis module 110 is present in a first unit 201, which may be a chamber like the chamber described above in the context of 101, and the dispensing module 120 and the sanitizing module 130 are present in a second unit 202, which may be a chamber like the chamber described above in the context of 101. The chambers of the first unit 201 and the second unit 202 in this example are separate chambers, and they may have different levels of cleanliness. Because of the modular aspect of this arrangement 200, in some examples the two units 201, 202 may be in different rooms in the same factory or facility. The different rooms may have the same environment, or different environments. For example, the chamber of the first unit 201 may have a Grade D environment, and the chamber of the second unit 202 may have a Grade C environment. In one example, reducing the requirements of the first unit 201 to a Grade D, instead of maintaining it at the same level as the second unit 202 at Grade C, may lower the burden and costs associated with operations.

In the modular arrangement 200 as described in FIG. 2, synthesis module 110 may be enclosed by a shield 180 (but need not always be), and the dispensing module 120 and the sanitizing module 130 may be collectively, as a packaging unit 210, enclosed by another shield 180 (but need not always be). In the example shown in FIG. 2, second unit 202 includes two packaging units 210, though other versions of second unit 202 may include more than two packaging units 210. The shields 180 of the synthesis module 130 and the packaging unit 210 may be the same or different composition or configuration. The sanitizing module 130 may contain a door that may be opened to receive the composition that has been dispensed. The production of pharmaceutical drug product that occurs in the first unit 201 and second unit 202 (and their corresponding modules contained therein) is carried out every time with consumables such as a new cassette 140, new set of reagents, and new tubing. All consumables are sterile and single use. In some examples, the compounding process as illustrated in FIG. 2 is carried out in dispensing module 120. The modular setup may have one or multiple dispensing units 120 which may be configured for dispensing to different receptacle types (e.g., vials, syringes, and the like) as described in more detail below.

In the arrangement 200 as shown in FIG. 2, a flow control module 203 may be employed to regulate at least one flow property of the fluid travelling between the first unit 201 and the second unit 202. The fluid may comprise a solution that contains the radiopharmaceutical composition leaving the first unit 201 travelling towards the second unit 202. The flow control module 203 may be employed to regulate the flow properties by any suitable mechanisms. For example, the flow control module 203 may contain certain hardware components that may be employed, individually or collectively, to regulate the fluid flow. The components may be pumps, valves, manifolds, or other hardware that may be used to regulate fluid flow. The flow control module 203 may regulate the flow by software, which may include any algorithm suitable to operate the mechanical components of the module to regulate fluid flow.

In contrast to the manual process as shown in FIG. 1, the process carried out in the dispensing module 120 in the process as shown in FIG. 2 may be automated. Any suitable algorithm and hardware, such as robotic elements, may be employed to automate the operations carried in the dispensing module 120. As a result, in this example arrangement 200 of FIG. 2, the number of operators 150 involved may be lower than that involved in the arrangement 100 in the process as shown in FIG. 1. For example, the number of operators 150 may be at least about 25% lower—e.g., at least about 50% lower, or about 100% lower.

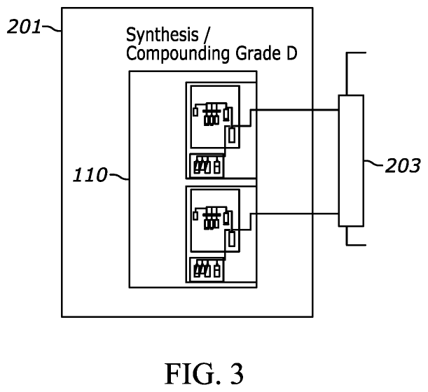

FIG. 3 illustrates, in one example, the processes taking place in a synthesis module 110. In one example, the process may include synthesizing, compounding, or both, the radiopharmaceutical composition. The processes and module may be any of those described herein, including those shown in FIG. 2. In one example, the operation of this synthesis module 110, similar to that shown in FIG. 2, may take place in a Grade D environment. As shown in FIG. 3, a flow control module 203, such as one described above, may be employed here.

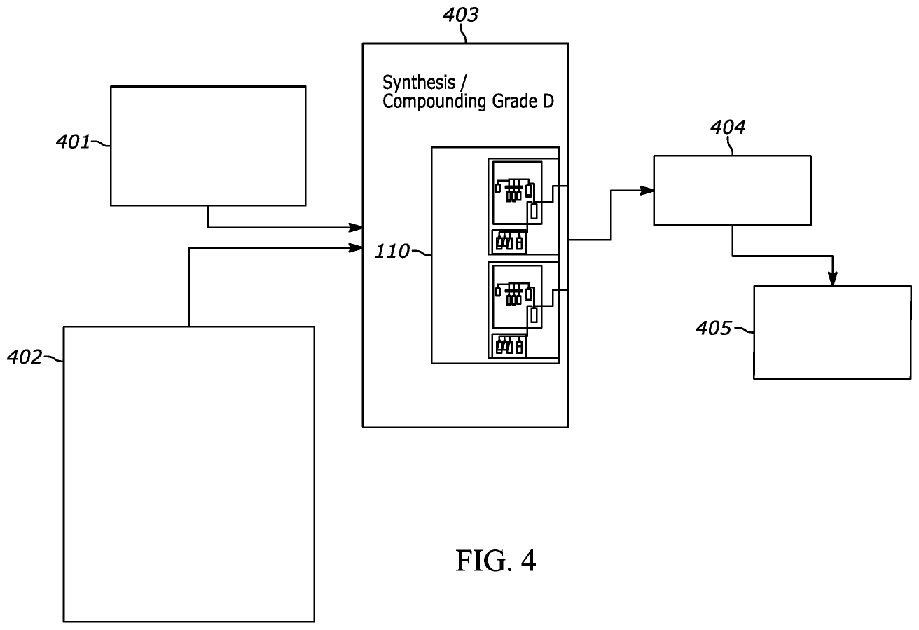

Table 1 summarizes at least some of the processes, including inputs and outputs, that can take place in synthesis module 110. Table 1 is described in further details with respect to FIG. 4 below.

TABLE 1

| | | Output (process |
|---|---|---|
| Input | WorkCenter | completion) |
| Kit Cassette | Pre-DS processes | Installed Kit Cassette |
| Peptide, Reaction, | Synthesis | DS |
| Buffer, WFI, Source | | |
| Dilution solution material | Cold Compounding | Dilution Solution |
| DS + Dilution Solution | Hot compounding/ | Hot Compound Solution |
| | Final Dilution | |

FIG. 4 further illustrates the processes that may take place in the synthesis module 110. As shown in FIG. 4, the cassette, such as cassette 140 shown in FIG. 2, may be employed for pre-drug substance (“DS”) processes, and then become installed into the synthesis module 110. As shown in process 401, peptide (i.e., ligand), reaction buffer, water for injection (“WFI”), and other source materials (e.g., radionuclide source) may be employed as part of the synthesis to produce at least part of the DS. In one example, activity measurement of source materials is performed 1 day before synthesis in dose calibrator, this activity is targeted in ILA. As shown in process 402, a dilution solution material may be employed to undergo cold compounding to produce at least part of the dilution solution. As shown in process 403, cold and hot dilution manufacturing may be employed. DS and dilution solution may be employed to undergo hot compounding and/or final dilution to produce at least part of the hot compound solution—in one example a diluted bulk mixture (process 404), which is a combination of the aforementioned materials, may then be transferred to the module configured to dispense (e.g., fill) the product into a packaging material. In process 405, the final diluted bulk mixture is transferred to the dispensing module. There need not be additional processes between process 404 and process 405, but additional processes, sch as sanitization, combination, etc., maybe be employed after process 404 to achieve process 405.

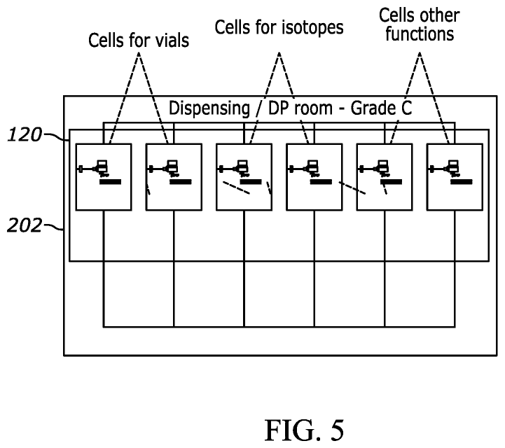

Table 2 summarizes at least some of the processes, including inputs and outputs, that can take place in the second unit 202, such as in the dispensing module 120. Table 2 is described in further details with respect to FIG. 5 below.

TABLE 2

| | | Output (process |
|---|---|---|
| Input | WorkCenter | completion) |
| Diluted Mixture | Transfer Line | Dispensed Vial |
| Kit, EM plate, | Filling and Closure | Filled vials or syringes |
| Vials, and Caps | | with stoppers closed |
| Filled Vial | Temporary shielded | Vial inside lead protected |
| | pack | tray |
| Residues from batch | Post batch controls | GIT and EM |

FIG. 5 illustrates the processes related to filling and closing that may take place in the second unit 202, for example in the dispensing module 120, as that shown in FIG. 2. The manufacturing environment may be the same as, or different from, that of the first unit 201 as shown in FIGS. 2 and 3 and described above. In one example, the environment of the second unit 202 is different from the first unit 201, and the second unit 202 has a Grade C environment. In one implementation, the processes described herein may be automated, as described herein. The diluted mixture may undergo a process in the transfer line to be dispensed into a packaging material, such as a vial or a syringe to form at least a part of dispensed vial. The syringe here may be a pre-filled syringe, such as a pre-filled glass syringe. In one example, the kit cassettes 140, environmental settle (“EM”) plate, vials, and caps may be employed to undergo processes related to filling and closure to produce at least part of filled vials or syringes with stopper closed. The filled vial may be placed in a temporary, shielded pack, which may then be placed in a lead protected tray. The residuals from the batch may undergo processes related to post-batch controls to produce at least part of GIT and EM. As shown in FIG. 5, this dispensing module 120 may further comprise hardware, or cells, dedicated for dispensing material into packaging materials (e.g., vials), or dispensing at least one radionuclide, which may be any of these described herein. For example, the radionuclide may be Lu-177. Alternatively, the radionuclide may be Ac-225. Additional hardware suitable for other functions may be present.

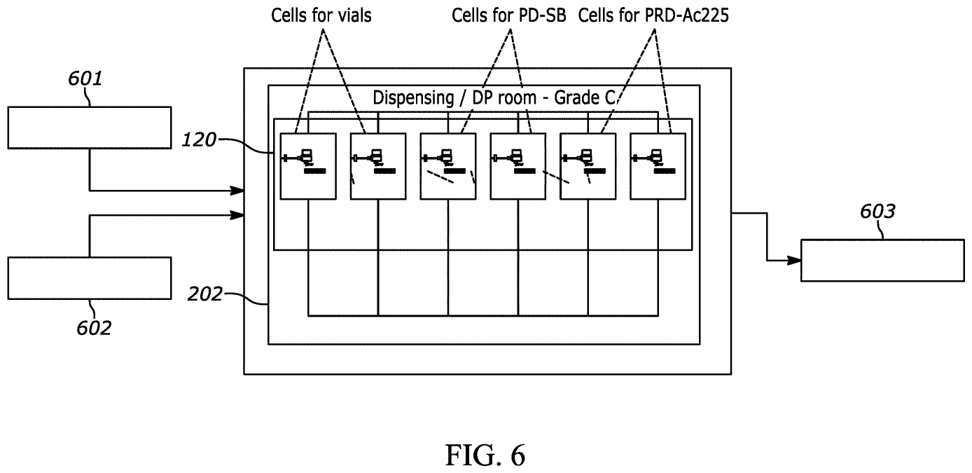

FIG. 6 further illustrates, in one example, additional processes that may be involved in the second unit 202. In this example, the modular process may be broken into three operations, and avoid dependency on single cell operations. As shown in process 601, the final diluted bulk drug product (such as that described in process 404 of FIG. 4) may be fed into the dispensing module 120. In process 602, sanitization, such as VHP (vaporized hydrogen peroxide) sterilization, may be employed. As part of this process, a dispenser kit cassette 140 and EM plate and packaging may be employed. In the dispensing unit as shown in FIG. 6, the dispensing module 120 may further comprise hardware, or cells, dedicated for dispensing material into packaging materials (e.g., vials), or dispensing into a patient ready device (“PRD”) such as a pre-filled syringe, to create filled patient doses (process 603). In one example, the system is configured for use with the radionuclide Ac-225. In another example, the system is configured for use with the radionuclide Lu-177. Additional hardware suitable for other functions may be present.

As noted above, the processes, alone or in combination, taking place in the dispensing module 120 of the second unit 202 may be automated. The automation may be achieved by any suitable mechanisms described herein.

Additional modification to those described herein have been contemplated. For example, with respect to the disposable cassettes 140, any suitable techniques to achieve desired level of cleanability, decontamination, radioactive safety, good manufacturing process (“GMP”) and usability may be employed. With respect to the flow control module 203, any suitable designs with respect to the mechanical hardware (e.g., pumps, valves, manifolds, robots) may be employed. Additionally, automation of liquid transfer to generate adequate product flows to a designated dispensing module 120 can be employed. The automation may be employed to reduce manual involvement; in some examples, it may remove manual involvement altogether. In one example, the process does not involve isolator gloves, such that all material preparation cell transfers, environmental settle plate placement and positioning may be automated. In some examples, the automation may take place outside of the cell in the module, or outside of the module. Suitable techniques to monitor and control product retention and process robustness to minimize lost batches and maximize output may be employed. Additionally, any suitable techniques to register the content of each packaged product (in a vial, syringe, etc.) and the traceability of the packaged product, such as a 2D matrix or QR code), may be employed.

One study has been performed to demonstrate the feasibility of the design designed herein, using different systems: Run 1, Run 2, and Run3 as shown in Table 3 below.

TABLE 3
Filling PoC Main Outcomes

| | Run 1 | Run 2 | Run 3 |
|---|---|---|---|
| Number of filled & | PoC 1: 700 units | PoC 1: 1000 units | PoC 1: 1050 units |
| stoppered syringes | PoC 2: 400 units | PoC 2: 200 units | Total: 1050 units |
| | Total: 1100 units | Total: 1200 units | |
| Failures due to | 2 suspects during | Few cases during | No contamination |
| contamination | PoC 1, but then | PoC 1 due to | observed |
| | confirmed to be due | incorrect filling | |
| | to air humidity. | parameters. | |
| | 2 cases during PoC 2, | No contamination | |
| | but due to manual | during PoC 2. | |
| | manipulation of caps | | |
| | (not leak tight after | | |
| | manipulation). | | |
| Failures due to | Some cases in PoC 1 | No failures | No failures |
| filling accuracy | due to wrong | | |
| | adjustment | | |
| | parameters on pump. | | |
| | When corrected, no | | |
| | more cases. | | |
| | No failures in PoC 2. | | |
| Failures due to | No failures | No failures | No failures |
| incorrect stoppering | | | |
| and/or bubble size | | | |
| Final outcome | PoC 1 released with | PoC 1 released with | Successful |
| | reserve. | reserve. | |
| | PoC 2 successful and | PoC 2 successful and | |
| | solved reserve on | solved reserve on | |
| | PoC 1. | PoC 1. | |
| Additional comments | Vacuum stoppering | Vented tube | Vacuum stoppering |
| | technology. | stoppering | technology. |
| | Technology | technology. Observed | Successful PoC but |
| | dimensions and type | risk of cross | technology difficult |
| | suitable to be | contamination in case | to be adjusted to fit |
| | adjusted to fit S.A. | of tube | S.A. Line spaces. |
| | Line spaces (already | contamination, as | |
| | used in table top | well as risk of scratch | |
| | units). | in case of tube | |
| | | misalignment. | |

With respect to the filling systems (e.g., in dispensing module 120), available technology suitable for RLT manufacturing processes has been contemplated. To manage syringes, suitable technology may be employed to improve accuracy, control, reliability and performance. This may lead to a not negligible increase in the cost as well as the needed space for the filling unit. It is possible to retrofit current lines with some compromises for ergonomy and automation level, but new lines may involve more space in the filling section to exploit the full potential of this technology. In some examples, combo functionality does not increase the complexity of the machine. Syringe filling and stoppering technology may cover the vial fill and finish technology, and therefore once the design and the process is suitable for syringes, it may be transferrable to vials with few format parts and disabling part of the functionalities.

Filling process may be important in terms of mitigating risk of radioactive contamination. Use of specialized peristaltic pumps with suitable circuits may mitigate the risk, but filling speed may become limited to a couple of syringes/vials per minute. Filling accuracy may be achieved in some implementions, but the bigger dimensions of the new filter may involve longer time for wetting, as well as a risk of filling accuracy reduction once air enters when bulk is emptied. A mitigation solution involving a pinch valve before the needle to stop the flow when the weight is reached may be employed. Stoppering mechanism has been developed to show that vacuum stoppering was suitable technology in terms of efficiency, performances, and complexity. The accuracy of the machine in positioning the stopper inside the vacuum chamber may be important to minimize contact between the stopper and the chamber. Mechanical stoppering (vented tube) may be suitable, but in some instances may introduce some risks linked to the alignment of the tube itself (risk of contamination or risk of particle creation) to be fully assessed. In the S.A. Line retrofit application, one manual operation is left, e.g., the stopper pre-seating (where an additional manual tool is to be designed and produced). In one implementation, additional primary packaging materials may be applied. In one example, this risk of delay as a result can be mitigated with a fully automated denesting system in future lines.

With respect to the discharge system, in some examples it was observed using the same system for vials and syringes may result in compromise of the mechanical design between the different geometries. In some examples, this may create risks for vials to break and for syringes to get stuck, but these risks, in some examples, may be mitigated by the customization of the barrel design (shorter and reduced flange), as well as some additional features introduced by TEMA in the design (e.g., Actuated centering rings, higher performance laser sensors, etc.). In one implementation, dual system syringe/vials (2 discharges) may be employed.

With respect to the Inspection, Labeling and Assembly, in some examples, available inspection technology (CCIT, VI and Dose Calibrator) may be employed for combo use, and devices may be doubled to minimize risks. In some implementations, final user processes multiple batches in the machine at the same time, and this may increase complexity from an ITOT perspective. Mitigation techniques have been contemplated. For CCIT, syringes may involve vacuum decay method. Due to absence of air in syringes, this method may lead to device vacuum chamber contamination at each failed test, which may involve entire vacuum chamber replacement. This may be mitigated by executing a vacuum chamber empty test before each CCIT, and the chamber itself may be shielded to allow safe removal in case of need. At the same time, the detectability of leakages may be high (estimated 5 to 10 times higher than vials), mitigating the risk of contaminated syringes to be delivered to hospitals. Label simplification (in particular with reduction of colors, or even black and white) has been contemplated and may be employed. In some implementations, combo functionality is not an additional challenge for labeling because syringe requirements in these examples may fully cover vial requirements. Assembly system design has been contemplated.

Modular RLT Manufacturing Arrangement with Transport Machines

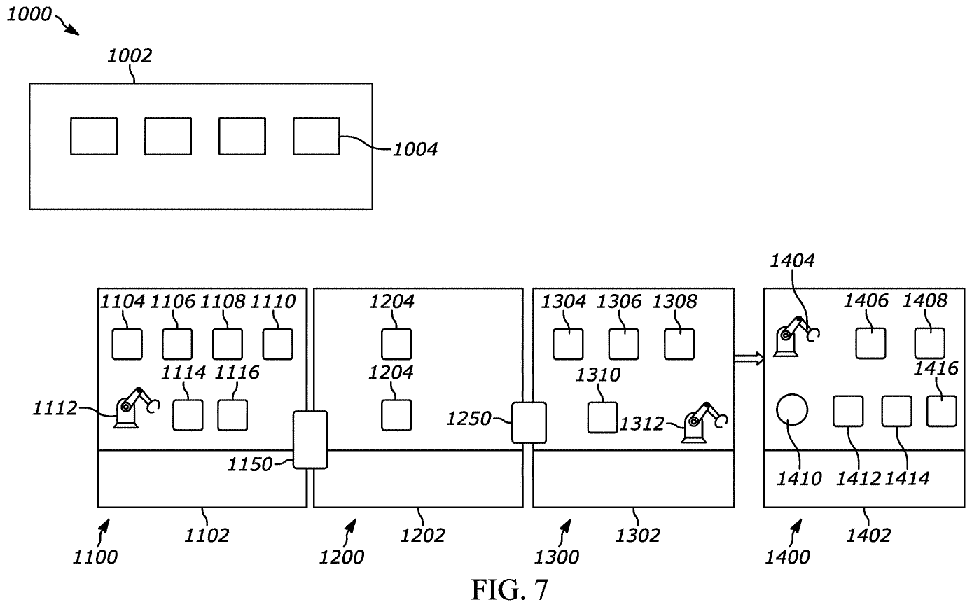

While arrangement 200 of FIG. 2 provides linking of units 201, 202 via flow control module 203, units of a modular RLT manufacturing process may be linked in other ways. For instance, FIG. 7 shows an arrangement 1000 where modules 1100, 1200, 1300, 1400 are linked via one or more transport machines 1204. In this arrangement 1000, personnel (e.g., one or more human operators) in a site control room 1002 oversee operation within and between modules 1100, 1200, 1300, 1400. Control room 1002 may include various kinds of equipment 1004 (e.g., one or more computers, etc.) that allow personnel to provide batch control, production planning, environmental monitoring, management of a fleet of transport machines 1204, manufacturing execution system (MES) management, management of therapeutic composition parameters, and/or other control over modules 1100, 1200, 1300, 1400. Control room 1002 may also provide personnel with full visibility of modules 1100, 1200, 1300, 1400. Such visibility may include direct visibility (e.g., via windows, etc.) and/or indirect visibility (e.g., via cameras, etc.).

Modules 1100, 1200, 1300, 1400 of this example include a synthesis and formulation module 1100, a transfer operation module 1200, a dispensing module 1300, and an inspection, labelling, and assembly (ILA) module 1400. Examples of features that may be included in each of these modules 1100, 1200, 1300, 1400 will be described in greater detail below. In the present example, a first rapid transfer port (RTP) 1150 is interposed between synthesis and formulation module 1100 and transfer operation module 1200. Similarly, a second RTP 1250 is interposed between transfer operation module 1200 and dispensing module 1300. In the present example, there is no RTP interposed between dispensing module 1300 and ILA module 1400, though other variations of arrangement 1000 may provide an RTP interposed between dispensing module 1300 and ILA module 1400.

Synthesis and formulation module 1100 may provide functionality similar to synthesis module 110 described above. As shown in FIG. 7, synthesis and formulation module 1100 of this example includes an enclosure 1102 containing various components. In some versions, enclosure 1102 includes shielding, such that synthesis and formulation module 1100 constitutes a shielded unit. In the present example, synthesis and formulation module 1100 is configured to provide a Grade C environment within enclosure 1102. One or more locking doors may permit entry into enclosure 1102 as needed. The components contained within enclosure 1102 include scales 1104, a diluent preparation stage 1106, a bulk handling stage 1108, a vaporized hydrogen peroxide (VHP) unit 1110, a robot 1112, a dose calibration stage 1114, and a synthesizer unit 1116.

Scales 1104 may include one or more scales that are operable to sense the mass of reagents and/or other substances that are used to synthesize and/or formulate RLT.

Diluent preparation stage 1106 may include any suitable components that may be used to prepare a diluent for use in the process of synthesizing and/or formulating RLT. While diluent preparation stage 1106 is shown as being within the same space within enclosure 1102 as the other components of synthesis and formulation module 1100, in other versions diluent preparation stage 1106 may be located in a different space (e.g., separated by one or more walls) relative to the other components of synthesis and formulation module 1100.

Bulk handling stage 1108 may include any suitable components that may be used to prepare or handle bulk materials for use in the process of synthesizing and/or formulating RLT. In some versions, bulk handling stage 1108 receives synthesized compositions from synthesizer unit 1116, such that the synthesized compositions from synthesizer unit 1116 may be gathered at bulk handling stage 1108. By way of further example only, bulk handling stage 1108 may be operable to automatically transfer a formulation, a diluent (e.g., from diluent preparation stage 1106) and/or a mother vial in bulk volumes.

VHP unit 1110 may include any suitable components that may be used to generate VHP, which may be used to provide sterilization during the process of synthesizing and/or formulating RLT. In the present example, VHP unit 1110 is operable to be started remotely. VHP unit 1110 is also operable to provide VHP to RTP 1150, to thereby maintain sterilization at RTP 1150.

Robot 1112 may include one or more robotic arms and/or other robotic features that may be used to handle materials and devices during the process of synthesizing and/or formulating RLT. For instance, robot 1112 may grasp, move, and release fluid containers and/or other items within enclosure 1102. Alternatively, robot 1112 may be used in any other suitable fashion. As used herein, the term “robot” term should be understood to include any suitable kind of robotic or otherwise mechanized apparatus that is capable of handling materials and devices during the process of synthesizing and/or formulating RLT, such that the term should not be dead as necessarily requiring an arm structure per se. While only one robot 1112 is shown, synthesis and formulation module 1100 may include two or more robots 1112.

Dose calibration stage 1114 may include any suitable components that are operable to calibrate dosages associated with the synthesized and formulated RLT.

Synthesizer unit 1116 may include any suitable components (e.g., a reactor, and reservoirs/receptacles for radionuclide source, ligand source, and reaction buffer, etc.) that are operable to perform a synthesis process as described herein. In some versions, synthesizer unit 1116 is an off the shelf synthesizer.

In the present example, all the above-noted components of synthesis and formulation module 1100 are capable of automated control. Such automated control may be initiated, coordinated, and/or overseen by personnel in control room 1002 or elsewhere. Personnel may directly interact with synthesis and formulation module 1100 in some scenarios, such as when setting up one or more kits for use in synthesis and formulation module 1100, to intervene if there is any kind of equipment failure within synthesis and formulation module 1100, etc. For instance, a fixed set of gloves (not shown) may pass through enclosure 1102 to allow personnel to handle components or materials within enclosure 1102 without the personnel coming into direct contact with such components or materials. In the present example, synthesis and formulation module 1100 provides an output in the form of a RLT drug, and may further output a bill of materials and/or other forms of output.

RTP 1150 of the present example includes a passageway that is formed between synthesis and formulation module 1100 and transfer operation module 1200. RTP 1150 is configured to allow materials (e.g., RLT in vials or other containers) to be transported from synthesis and formulation module 1100 to transfer operation module 1200. RTP 1150 may include at least one door on each end of the passageway, with each door being configured to seal the corresponding end of the passageway, such that RTP 1150 provides an airlock configuration. RTP 1150 may thus maintain aseptic conditions within the interior of RTP 1150, even as materials (e.g., RLT in vials or other containers) are transported from synthesis and formulation module 1100 to transfer operation module 1200. As noted above, VHP unit 1110 may be operable to provide VHP to RTP 1150, to thereby maintain sterilization at RTP 1150.

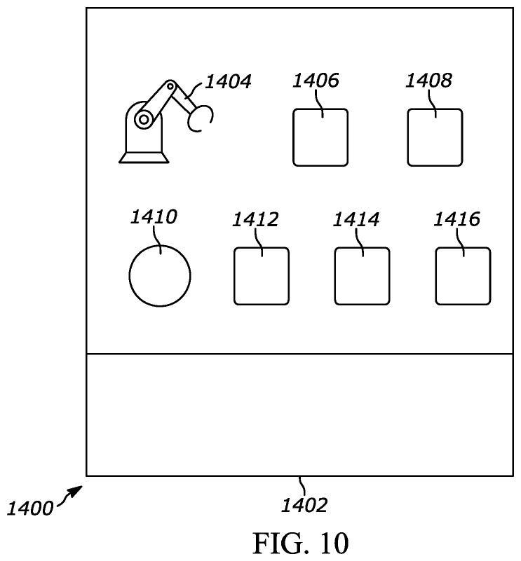

FIG. 10 shows another variation of synthesis and formulation module 1101 that may be incorporated into arrangement 1000 in place of synthesis and formulation module 1100. Like synthesis and formulation module 1100, synthesis and formulation module 1101 of this example includes an enclosure 1103, a diluent preparation stage 1107, a bulk preparation stage 1109, a VHP unit 1111, a robot 1112, and a synthesizer unit 1117. Synthesis and formulation module 1101 of this example further includes a glove tester 1121 and a transfer system 1123. Glove tester 1121 may include components (e.g., an inflator and leak detector) that are operable to test the integrity of one or more gloves (e.g., one or more gloves that pass through enclosure 1102 to allow personnel to handle components or materials within enclosure 1102).

Transfer system 1123 may include one or more components that are configured to facilitate transfer of RLT from synthesis and formulation module 1101 to transfer operation module 1200 via RTP 1150. In addition, or in the alternative, transfer system 1123 may be operable to transfer synthesized compositions from synthesizer unit 1117 to bulk preparation stage 1109. In addition, or in the alternative, transfer system 1123 may be operable to transfer synthesized compositions from bulk preparation stage 1109 to transfer operation module 1200 via RTP 1150. By way of further example only, transfer system 1123 may include fluid conduits, pumps, valves, fluid transfer vessels, and/or any other suitable components. In some versions, transfer system 1123 provides user input features (e.g., a human machine interface (HMI) features) that allow personnel to control such pumps, valves, etc. It should be understood that synthesis and formulation module 1101 may also include numerous other user input features (e.g., to open doors, start processes, etc.), despite synthesis and formulation module 1101 being largely subject to automated control.

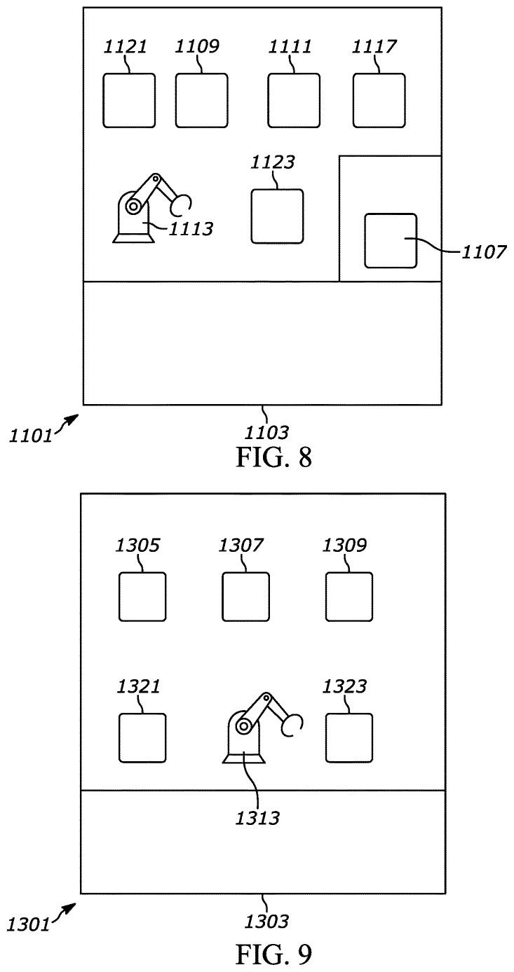

While not shown in FIG. 8, synthesis and formulation module 1101 may also include scales, a dose calibration unit, and an RTP, respectively like scales 1104, dose calibration stage 1114, and RTP 1150 of synthesis and formulation module 1100. Similarly, synthesis and formulation module 1100 may include glove tester 1121 and transfer system 1123. Either synthesis and formulation module 1100, 1101 may include numerous other components, in addition to or in lieu of any of the components described above.

As shown in FIG. 7, transfer operation module 1200 of this example includes one or more transport machines 1204 within an enclosure 1202, and an RTP 1250. In some versions, enclosure 1202 includes shielding, such that transfer operation module 1200 constitutes a shielded unit. Transfer operation module 1200 may be configured to provide any suitable Grade (e.g., Grade A, Grade B, Grade C) environment within enclosure 1202. One or more locking doors may permit entry into enclosure 1202 as needed. While two transport machines 1204 are shown in FIG. 7, any suitable number of transport machines 1204 may be provided, including just one or more than two. Each transport machine 1204 may take any of various forms. In some versions, each transport machine 1204 includes two or more wheels, treads, or other drivable features; and is capable of moving along a floor and/or other surface(s) within enclosure 1202. In some versions, each transport machine 1204 is configured to move along a set of rails like a railcar, along a wire, along a magnetic strip, along an optical path, or along some other fixed route. Alternatively, transport machine 1204 may be configured to move along any ad hoc path to get from one point (e.g. RTP 1150) to another point (e.g. RTP 1250). Each transport machine 1204 may comprise an autonomous guided vehicle, an autonomous mobile robot, or any other suitable kind of device. It should therefore be understood that transport machines 1204 may be controlled automatically, including their movement within enclosure 1202 and their interactions with RTPs 1150, 1250.

Each transport machine 1204 is further configured to retrieve materials (e.g., RLT in vials or other containers) from RTP 1150 and transfer those materials to RTP 1250. By way of example only, each transport machine 1204 may include one or more robotic arms and/or other features that can retrieve materials from RTP 1150 and deposit those materials at RTP 1250 after transport machine 1204 traverses a space between RTPs 1150, 1250. Each transport machine 1204 may further include a compartment or other space to hold those materials en route from RTP 1150 to RTP 1250. Alternatively, transport machine 1204 may simply hold the materials in the same robotic arm or other feature that is used to retrieve and deposit the materials from RTP 1150 and at RTP 1250. As another variation, one or more robotic arms and/or other features may be integrated at each RTP 1150, 1250. In such versions, the robotic arm and/or other feature at RTP 1150 may deposit the materials on or in a transport machine 1204, the transport machine 1204 may then traverse the space from RTP 1150 to RTP 1250, and then the robotic arm and/or other feature at RTP 1250 may retrieve the transported material at RTP 1250.

RTP 1250 may be configured and operable like RTP 1150 described above. While only one RTP 1250 is shown in FIG. 7, some variations may provide more than one RTP 1250 and more than one dispensing module 1300, where an RTP 1250 is associated with each respective dispensing module 1300. Thus, transport machines 1204 may retrieve materials from RTP 1150 and deliver those materials to any of the various dispensing modules 1300 via the RTP 1250 associated with a target dispensing module 1300. After delivering materials to RTP 1250, a transport machine 1204 may return to RTP 1150 to retrieve more materials from synthesis and formulation module 1100 or may park at an appropriate location within enclosure 1202 until transport machine 1204 is needed.

While transport machines 1204 operate within enclosure 1202 in this example, some variations may provide movement of transport machines 1204 within a non-enclosed space. In such versions, transport machines 1204 may include shielded containment space, and RTPs 1150, 1250 may include shielding, such that materials may be safely transported from synthesis and formulation module 1100 to dispensing module 1300 via transport machines 1204 without providing radioactive contamination of the space outside of the shielded containment space of transport machine 1204 and RTPs 1150, 1250. In other words, transport machine 1204 and RTPs 1150, 1250 may maintain shielding of the materials from synthesis and formulation module 1100 during transport of the materials from synthesis and formulation module 1100 to dispensing module 1300.

In the present example, transport machines 1204, the vessels containing materials from synthesis and formulation module 1100, and RTPs 1150, 1250 may collectively provide a function similar to that provided by flow control module 203 in arrangement 200. In other words, the products of synthesis and formulation module 1100 may be selectively distributed to various dispensing modules 1300. In cases where one dispensing module 1300 fails, needs maintenance, or is otherwise not in condition to receive materials from synthesis and formulation module 1100, transfer operation module 1200 may simply deliver the next batch of materials from synthesis and formulation module 1100 to another dispensing module 1300.

In cases where synthesis and formulation module 1100 is capable of outputting materials at a rate that is faster than the handling rate of a single dispensing module 1300, transfer operation module 1200 may distribute materials from synthesis and formulation module 1100 to numerous dispensing module 1300 such that two or more dispensing modules 1300 may process those materials simultaneously or otherwise in parallel. As yet another example, synthesis and formulation module 1100 may be configured to generate various different types of outputs, and different dispensing modules 1300 may be specially configured to handle different types of outputs from synthesis and formulation module 1100. In such scenarios, transfer operation module 1200 may distribute materials from synthesis and formulation module 1100 to the appropriate dispensing module(s) 1300 based on a match between the type of output from synthesis and formulation module 1100 at hand. Alternatively, the routing capabilities of transfer operation module 1200 may be utilized in any other suitable fashion.

Dispensing module 1300 may provide at least some features and functionalities like those described above in the context of dispensing module 120. As shown in FIG. 7, dispensing module 1300 of this example includes an enclosure 1302 containing various components. In some versions, enclosure 1302 includes shielding, such that dispensing module 1300 constitutes a shielded unit. In the present example, dispensing module 1300 is configured to provide a Grade A environment within enclosure 1302. One or more locking doors may permit entry into enclosure 1302 as needed. The components contained within enclosure 1302 include a bulk handling stage 1304, scales 1306, a dispensing stage 1308, a primary packaging stage 1310, and a robot 1312.

Bulk handling stage 1304 may include any suitable components that may be used to prepare or handle bulk materials for use in the process of dispensing RLT in dispensing module 1300. For instance, a bulk volume of RLT composition may be received in dispensing module 1300 via RTP 1250, and this bulk volume of RLT composition may be held in bulk handling stage 1304 while other components of dispensing module 1300 dispense the RLT composition into vessels (e.g., vials) that have a smaller capacity than a bulk container (e.g., flask or bottle) within bulk handling stage 1304.

Scales 1306 may include one or more scales that are operable to sense the mass of RLT compositions and/or other substances that are handled within dispensing module 1300. By way of example only, one or more scales 1306 may be used to track the volume of RLT composition within bulk handling stage 1304. By way of further example only, one or more scales 1306 may be used to measure aliquots of the RLT composition from bulk handling stage 1304 for further processing in dispensing stage 1308. By way of further example only, one or more scales 1306 may be used to sense the mass of a vessel (e.g., vial) that has been filled with RLT composition by dispensing stage 1308 before the vessel is transported out of dispensing module 1300. Alternatively, scales 1306 may be used in any other suitable fashion.

Dispensing stage 1308 may include any suitable components for dispensing RLT composition from bulk handling stage 1304 into smaller vessels (e.g., vials). Dispensing stage 1308 may thus include one or more fluid conduits, pumps, valves, etc. In the present example, dispensing stage 1308 is operable to automatically dispense a predetermined or programmed volume of RLT composition into each vessel. Dispensing stage 1308 may be further operable to seal and/or otherwise enclose the RLT composition within the vessel, such as by using stoppers, shielding, and/or other components. By way of example only, dispensing stage 1308 may include one set of features that are operable to provide a fluid seal of RLT within the vessel (e.g., by inserting a stopper into a vial, etc.); and another set of features that are operable to position the sealed vessel in a shield assembly (i.e., to contain radiation from the sealed vessel). Alternatively, dispensing module 1300 may include a separate stage that positions one or more sealed vessels in a shield assembly.

Robot 1312 may include one or more robotic arms and/or other robotic features that may be used to handle materials and devices during the process of dispensing RLT in dispensing module 1300. For instance, robot 1312 may grasp, move, and release fluid containers and/or other items within enclosure 1302. By way of example only, robot 1312 may provide movement that is part of the process of transferring RLT from a container received via RTP 1250 into bulk handling stage 1304. By way of further example only, robot 1312 may provide movement that is part of the process of transferring RLT from bulk handling stage 1304 to dispensing stage 1308, though any other suitable components or techniques may be used to transfer transferring RLT from bulk handling stage 1304 to dispensing stage 1308. By way of further example only, robot 1312 may provide movement that is part of the process of transferring a vessel to and/or from dispensing stage 1308. Alternatively, robot 1312 may be used in any other suitable fashion. While only one robot 1312 is shown, dispensing module 1300 may include two or more robots 1312.

Primary packaging stage 1310 may include a set of vessels that are to be filled by dispensing stage 1308. Such vessels may take any suitable form, including but not limited to vials, syringe barrels, etc. As noted above, robot 1312 may retrieve a vessel from primary packaging stage 1310, appropriately position the vessel in relation to dispensing stage 1308, hold the vessel while the vessel is filled with the appropriate volume of RLT by dispensing stage 1308, then move the filled vessel away from dispensing stage. In some versions, a plurality of vessels are grouped together in respective racks, trays, tubs, and/or other devices within primary packaging stage 1310. In some such versions robot 1312 may manipulate vessels collectively in groups, such as by grasping a racks, trays, tubs, and/or other device that holds a group of vessels together.

While not shown in FIG. 7, dispensing module 1300 may include a VHP unit (e.g., like VHP unit 1110), a glove tester (e.g., like glove tester 1121), and/or various other suitable components.

In the present example, all the above-noted components of dispensing module 1300 are capable of automated control. Such automated control may be initiated, coordinated, and/or overseen by personnel in control room 1002 or elsewhere. Personnel may directly interact with dispensing module 1300 in some scenarios, such as when setting up one or more kits for use in dispensing module 1300, to intervene if there is any kind of equipment failure within dispensing module 1300, etc. For instance, a fixed set of gloves (not shown) may pass through enclosure 1302 to allow personnel to handle components or materials within enclosure 1302 without the personnel coming into direct contact with such components or materials.

FIG. 9 shows another variation of dispensing module 1301 that may be incorporated into arrangement 1000 in place of dispensing module 1300. Like dispensing module 1300, dispensing module 1301 of this example includes an enclosure 1302, a bulk handling stage 1305, scales 1307, a dispensing stage 1309, and a robot 1313. Dispensing module 1301 of this example further includes a transfer system 1321 and a vessel extraction system 1323.

Transfer system 1321 may include one or more components that are configured to facilitate transfer of RLT from an RTP like RTP 1250 to bulk handling stage 1305. In addition, or in the alternative, transfer system 1321 may be operable to transfer RLT from bulk handling stage 1305 to dispensing stage 1309. By way of further example only, transfer system 1321 may include fluid conduits, pumps, valves, fluid transfer vessels, and/or any other suitable components. In some versions, transfer system 1321 provides user input features (e.g., a human machine interface (HMI) features) that allow personnel to control such pumps, valves, etc. It should be understood that dispensing module 1301 may also include numerous other user input features (e.g., to open doors, start processes, etc.), despite dispensing module 1301 being largely subject to automated control.

Vessel extraction system 1323 may include one or more components that are configured to provide extraction of filled vessels (e.g., vials, syringe barrels, etc.) from dispensing stage 1309. In addition, or in the alternative, vessel extraction system 1323 may include one or more components that are configured to provide extraction of filled vessels (e.g., vials, syringe barrels, etc.) from dispensing module 1301 for subsequent handling outside of enclosure 1303. By way of example only, vessel extraction system 1323 may include an automated conveyor belt or other continuous conveying apparatus that conveys filled vessels from dispensing module 1301 to another module, such as ILA module 1400 or elsewhere. In some versions, robot 1312 transfers filled vessels from dispensing stage 1309 to vessel extraction system 1323. Vessel extraction system 1323 may convey filled vessels individually or in groups (e.g., in trays, containers, or other devices holding two or more filled vessels together in a group). In the present example, the vessels are shielded by a shield assembly while being transported by vessel extraction system 1323. In addition, or in the alternative, vessel extraction system 1323 may include integral shielding to contain radiation from the filled vessels.

While not shown in FIG. 9, dispensing module 1301 may also include a primary packaging stage like a primary packaging stage 1310 of dispensing module 1300. Similarly, dispensing module 1300 may include transfer system 1321 and/or vessel extraction system 1323. Either dispensing module 1300, 1301 may include numerous other components, in addition to or in lieu of any of the components described above.

In the example shown in FIG. 7, there is no RTP interposed between dispensing module 1300 and ILA module 1400. This is because the RLT composition is contained in one or more vessels that is/are further contained in a shield assembly by the time the RLT composition exits dispensing module 1300. In some other versions, this vessel shielding is not yet provided in dispensing module 1300. In some such versions, the one or more vessels containing the RLT composition may be transferred from the dispensing module 1300 to the ILA module 1400 via a set of RTPs (e.g., one RTP at an exit of dispensing module 1300 and another RTP at an entrance of ILA module 1400). Transport machines like transport machines 1204 may be used to provide such transfer, and such transfer may take place within an enclosure like enclosure 1202 that provides a sealed and shielded environment between the exit RTP of dispensing module 1300 and the entry RTP of ILA module 1400.

As shown in FIGS. 7 and 10, ILA module 1400 of this example includes an enclosure 1402 containing various components. In some versions, enclosure 1402 includes shielding, such that ILA module 1400 constitutes a shielded unit. In the present example, ILA module 1400 is configured to provide a Grade C environment within enclosure 1402. One or more locking doors may permit entry into enclosure 1402 as needed. The components contained within enclosure 1402 include a robot 1404, a visual inspection stage 1406, a closed container integrity tester (CCIT) stage 1408, a dose calibration stage 1410, an assembly stage 1412, a shield labeling stage 1414, and a vessel labeling stage 1416.

Robot 1404 may include one or more robotic arms and/or other robotic features that may be used to handle materials and devices during the process of inspecting, labeling, and assembling in ILA module 1400. For instance, robot 1404 may grasp, move, and release vessels, shield assemblies, and/or other items within enclosure 1402. By way of example only, robot 1404 may provide movement that is part of the process of transferring items (e.g., a vessel containing RLT composition, enclosed within a shield assembly) from dispensing module 1300 into ILA module 1400. By way of further example only, robot 1404 may provide movement that is part of the process of moving an item into and out of visual inspection stage 1406. By way of further example only, robot 1404 may provide movement that is part of the process of moving an item into and out of CCIT stage 1408. By way of further example only, robot 1404 may provide movement that is part of the process of moving an item into and out of dose calibration unit. By way of further example only, robot 1404 may provide movement that is part of the process of moving an item into and out of assembly stage. By way of further example only, robot 1404 may provide movement that is part of the process of moving an item into and out of shield labeling stage. By way of further example only, robot 1404 may provide movement that is part of the process of vessel labeling stage. Alternatively, robot 1404 may be used in any other suitable fashion. While only one robot 1404 is shown, ILA module 1400 may include two or more robots 1404.

Visual inspection stage 1406 may include one or more cameras, one or more machine vision sensors, and/or other components operable to provide automated visual inspection of items. In cases where the vessel enters ILA module 1400 in a shield assembly, the vessel may be removed from the shield assembly (e.g., by robot 1404) before being placed in the visual inspection stage 1406. When the vessel is placed in the visual inspections stage 1406, visual inspection stage 1406 may capture one or more images of the vessel and/or the RLT composition within the vessel, and software may process the images to determine whether the optically observable qualities of the vessel and/or the RLT composition meet specifications. In some cases, visual inspection stage 1406 may also provide visual inspection of the shield assembly (e.g., after the already-inspected vessel is placed into the shield assembly). Alternatively, visual inspection stage 1406 may operate in any other suitable fashion.

In cases where the vessel enters ILA module 1400 in a shield assembly, the vessel may be removed from the shield assembly (e.g., by robot 1404) before being placed in CCIT stage 1408. CCIT stage 1408 may include components that are operable to subject the vessel containing RLT to a sealed environment where high pressure and/or vacuum is applied to the vessel. CCIT stage 1408 may further include components that are operable to detect whether there are any leaks from or into the vessel when the vessel is subject to the high pressure and/or vacuum. CCIT stage 1408 may thus be used to test the integrity of the closure of the vessel (e.g., as provided by a stopper inserted into the vial at the dispensing stage 1308, 1309 of dispensing module 1300, 1301.

In cases where the vessel enters ILA module 1400 in a shield assembly, the vessel may be removed from the shield assembly (e.g., by robot 1404) before being placed in dose calibration stage 1410. Dose calibration stage 1410 may include any suitable components that are operable to calibrate dosages associated with the synthesized and formulated RLT. In some cases, dose calibration stage 1410 of ILA module 1400 is configured and operable like dose calibration stage 1114 of synthesis and formulation module 1100.

Assembly stage 1412 may include components that are operable to provide assembly, and in some cases disassembly, of parts. For instance, assembly stage 1412 may include automated components that are operable to place the vessel containing RLT within a shield assembly. By way of example only, assembly stage 1412 may be operable to place the vessel containing RLT within a shield assembly after the vessel has passed through stages 1406, 1408, 1410, 1416 of ILA module; before or after the shield assembly has passed through shield labeling stage 1414. In cases where the vessel enters ILA module 1400 in a shield assembly, some versions of assembly stage 1412 may be operable to remove the vessel from the shield assembly, to allow the vessel to then pass through one or more stages 1406, 1408, 1410, 1416 of ILA module. As yet another example, assembly stage 1412 may be operable to place one or more shield assemblies that contain respective vessels of RLT in another shielded container (e.g., a case with a lid) for subsequent handling. Alternatively, assembly stage 1412 may operate in any other suitable fashion.

Shield labeling stage 1414 may include any suitable components that may be used to apply a label to a shield assembly that contains the vessel that contains the RLT composition. Such a label may include any suitable kind of information, including but not limited to an identification of the composition contained within the vessel, data indicating the date and/or other information associated with when the composition was created, the date and/or other information associated with when the vessel was filled, the date and/or other information associated with when the vessel was placed in the shield assembly, etc. The label may include a QR code, a bar code, or some other machine-readable indicia. In some versions, a label is first applied to the vessel by vessel labeling stage 1416 as described below, then the labeled vessel is enclosed in the shield assembly, and then a label is applied to the shield assembly by shield labeling stage 1414. In some other versions, shield labeling stage 1414 may apply a label to the shield assembly in parallel with the vessel labeling stage 1416 applying a label to the vessel. In any case, one or more of robots 1404 may move the shield assembly into and out of the shield labeling stage 1414.

Vessel labeling stage 1416 may include any suitable components that may be used to apply a label to a vessel (e.g., a sealed vial or syringe barrel, etc.). Such a label may include any suitable kind of information, including but not limited to an identification of the composition contained within the vessel, data indicating the date and/or other information associated with when the composition was created, the date and/or other information associated with when the vessel was filled, etc. The label may include a QR code, a bar code, or some other machine-readable indicia. As noted above, the vessel and the shield assembly may be respectively labeled in a sequence or in parallel. In any case, one or more of robots 1404 may move the vessel into and out of the vessel labeling stage 1416.

While the above-noted stages 1406, 1408, 1410, 1412, 1414, 1416 of ILA module 1400 are described in a certain sequence above, the stages 1406, 1408, 1410, 1412, 1414, 1416 of ILA module 1400 may be operated in any suitable sequence. For instance, a vessel containing RLT, with or without an enclosing shield assembly, may pass through stages 1406, 1408, 1410, 1412, 1416 in any suitable sequence. While ILA module 1400 is described in the context of arrangement 1000, an ILA module 1400 may also be incorporated into arrangement 200. For instance, the output of second unit 202 may be sent to an ILA module 1400 for further processing as described above. In any case, the output from the ILA module 1400 may include one or more labeled shield assemblies containing labeled vessels of RLT. Such output from the ILA module 1400 may then be sent through further processing; or may be sent out to a hospital or clinic, etc., for a physician to administer the RLT to a patient. In some cases, the output from the ILA module 1400 is further processed through a shipping module, such as shipping module 1516 described below.

### Information Processing

Utilizing the arrangements 200, 1000 described above may require a substantial amount of information processing. This may include information being processed at the various components of arrangement 200, 1000, information being exchanged between various components of arrangement 200, 1000, and information being exchanged with devices that are outside of arrangement 200, 1000. It may therefore be desirable to optimize efficiency in the information processing carried out during utilization of RLT manufacturing and packaging arrangements such as arrangements 200, 1000. The following provides an example of an arrangement, method of operation, and data flow that may enhance the efficiency in the processing of information during utilization of RLT manufacturing and packaging arrangements such as arrangements 200, 1000.

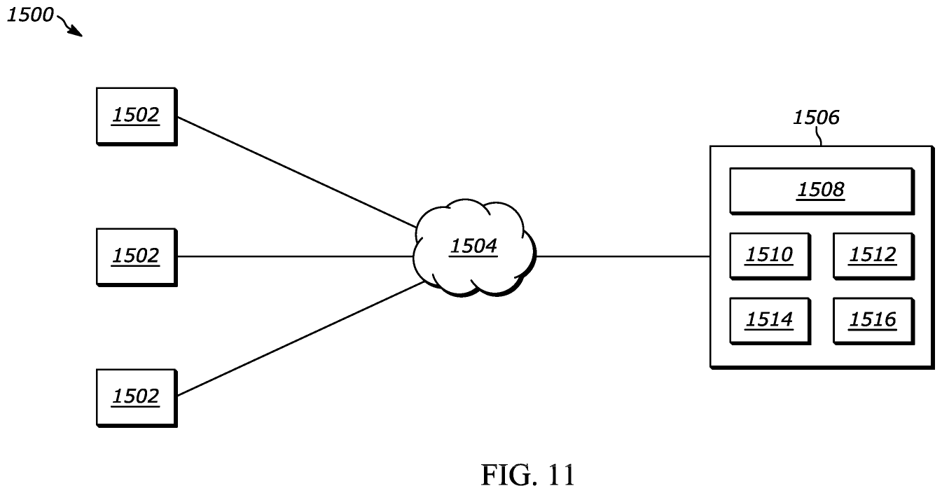

FIG. 11 shows an example of an arrangement 1500 where a plurality of remote computing devices 1502 are coupled with a radiopharmaceutical manufacturing system 1506 via a network 1504. Remote computing device 1502 may include desktop computers, laptop computers, tablets, smartphones, or any other kind of device that is operable to transmit information (e.g., commands, data, etc.) over network 1504. In some cases, one or more remote computing devices 1502 are located in a hospital, a physician's office, or some other location. Remote computing devices 1502 may thus be used by customers who wish to order radiopharmaceutical compositions for manufacture by radiopharmaceutical manufacturing system 1506.

Network 1504 may include the internet, a closed local network, or any other suitable kind of arrangement that is operable to provide transmission of information from each remote computing device 1502 to radiopharmaceutical manufacturing system.

Radiopharmaceutical manufacturing system 1506 includes a computer system 1508, a synthesis module 1510, a dispensing module 1512, an ILA module 1514, and a shipping module 1516. While only one a synthesis module 1510, one dispensing module 1512, one ILA module 1514, and one shipping module 1516 are shown, radiopharmaceutical manufacturing system 1506 may include numerous synthesis modules 1510, numerous dispensing modules 1512, numerous ILA modules 1514, and/or numerous shipping modules 1516. In some cases, having more than one module 1510, 1512, 1514, 1516 may provide redundancies to ensure continuous operation of radiopharmaceutical manufacturing system 1506 in the event that one or more modules 1510, 1512, 1514, 1516 fail or otherwise need to stop operating for any period of time. For instance, RLT compositions synthesized by just one synthesis module 1510 may be routed to various different dispensing modules 1512.

Computer system 1508 may include any suitable number and type(s) of computing devices, including processors and memory devices, etc.) that are operable to store and execute software as described herein; to thereby execute the methods described herein. Synthesis module 1510 is operable to synthesize an RLT composition. By way of example only, synthesis module 1510 may be configured and operable like synthesis module 110, may be configured and operable like synthesis and formulation module 1100, or may have any other suitable configuration and operability. Dispensing module 1512 is operable to dispense an RLT composition into a vessel. By way of example only, dispensing module 1512 may be configured and operable like dispensing module 120, may be configured and operable like synthesis and formulation module 1300, 1301, or may have any other suitable configuration and operability. ILA module 1514 is operable to provide inspection, labelling, and assembly associated with a vessel containing an RLT composition. By way of example only, RLT module 1514 may be configured and operable like ILA module 1400 or may have any other suitable configuration and operability. Shipping module 1516 is operable to perform tasks associated with shipping RLT products from ILA module 1400 to the customer (e.g., physician, hospital, etc.), and may include any suitable components that are configured an operable to accomplish such tasks.

Radiopharmaceutical manufacturing system 1506 may include one or more of the other components of arrangement 200, one or more of the other components of arrangement 1000, and/or any other suitable kind of arrangement that is operable to package radiopharmaceutical compositions; and in some cases, also synthesize/formulate the radiopharmaceutical compositions. Thus, while arrangement 1500 and the methods below are described herein in the context of arrangements 200, 1000, it is contemplated that arrangement 1500 and the methods below may also be utilized with other kinds of radiopharmaceutical manufacturing systems. The below teachings are thus not limited to arrangements 200, 1000.

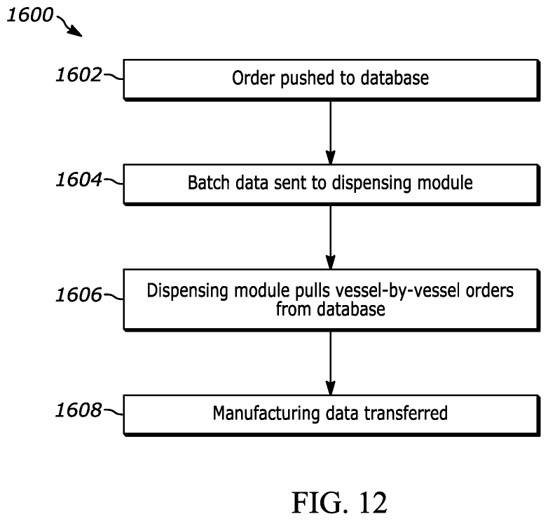

FIG. 12 shows an example of a process 1600 that may be carried out via arrangement 1500. Process 1600 and arrangement 1500 may be employed to provide an automated planning approach for the manufacture of radiopharmaceutical compositions that leverages serialization solutions. Process 1600 and arrangement 1500 may tend to remove a dependency on batch-level planning for customer orders for radiopharmaceutical compositions, where such dependency may otherwise be present in conventional processes. Process 1600 and arrangement 1500 may enable orders for radiopharmaceutical compositions to be filled by one or more dispensing modules of arrangement 1500 (e.g., dispensing module 120, 1300, 1301). Process 1600 and arrangement 1500 may enable such orders to be filled based on various predefined rules, including but not limited to date/time, distance to customer, shipping logistics, and/or other rules.

In this example, the process 1600 begins with an order being pushed to a database (block 1602). The order may be placed by a customer via remote computing device 1502, such that radiopharmaceutical manufacturing system 1506 may receive the order via network 1504. The remote computing device 1502 may provide access to an ordering portal at remote computing device 1502. In the present example, the order is pushed from an enterprise resource planning (ERP) software to the database. The database may be stored in a cloud (e.g., on a server that is remote from arrangement 1500), stored locally (e.g., on computer system 1508), or both.

Next, batch data is sent to dispensing module 1512 (block 1604). The batch data may be sent (block 1604) from computer system 1508 and/or any other suitable source. In some versions, the batch data is sent to dispensing module 1512 (block 1604) by a manufacturing execution system (MES), which may include software stored on and executed by computer system 1508 to produce electronic batch manufacturing records. In some cases, this batch data does not include data about specific vessels (e.g., vials) that are to contain RLT composition in accordance with the order.

Next, each dispensing module 1512 may pull individual customer orders from the database (block 1606), on a vessel-by-vessel basis. This may allow dispensing module 1512 to dispense and package RLT compositions ad hoc, tailoring each dispensation based on specific orders from customers.

Next, the manufacturing data may be transferred (block 1608) to a database. This data transfer may be executed from computer system 1508, from one or more modules 1510, 1512, 1514, 1546, and/or from any other suitable component of radiopharmaceutical manufacturing system 1506. The database receiving the transferred information may be stored in a cloud (e.g., on a server that is remote from arrangement 1500), stored locally (e.g., on computer system 1508), or both. The transferred manufacturing data may include whatever manufacturing data is required for release certificates. In some cases, the transferred data is eventually integrated into the ERP system.

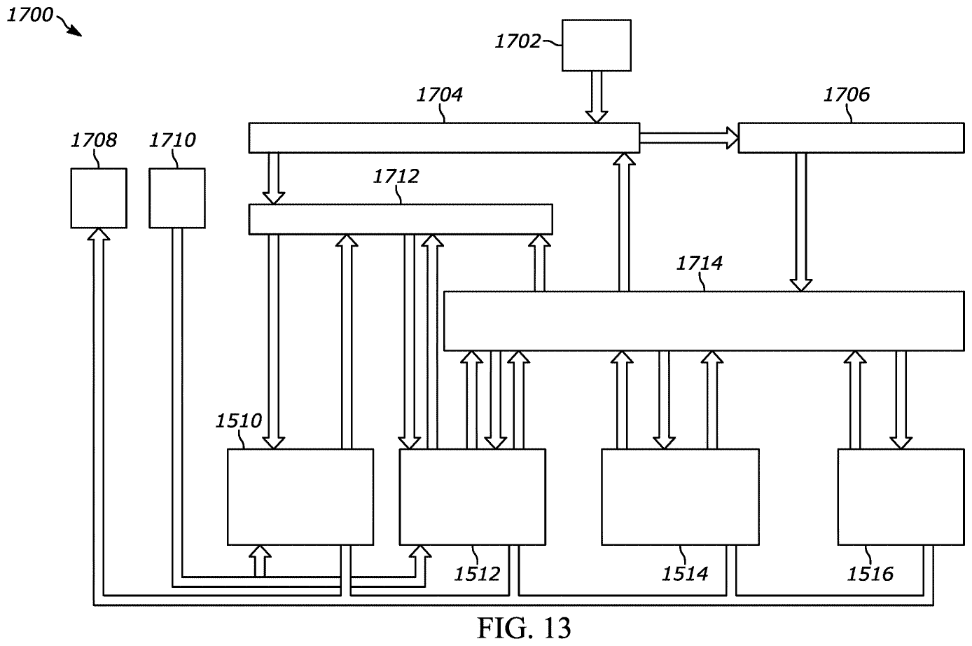

FIG. 13 shows an example of a data flow 1700 that may be carried out in an implementation of the process 1600 described above, using arrangement 1500. In this example, an order is received through ordering system 1702. As noted above, ordering system 1702 may be provided via a remote computing device 1502, network 1504, and computing system 1508. The order from ordering system 1702 reaches an ERP system 1704, which may reside on computing system 1508. This order may be pushed from ordering system 1702 to ERP system 1704 as described above with respect to block 1602.

After receiving the order, ERP system 1704 sends customer order details to a logistics planning module 1706, which may also be part of computing system 1508. Logistics planning module 1706 relays customer order details to a serialization system 1714, which may be part of computing system 1508. By way of example only, logistics serialization system 1714 may track the vessel (e.g., vial) containing RLT composition through various processes (e.g., dispensing, inspection/labeling, packaging, etc.) via a pre-printed serialized two-dimensional code (or other indicia). Serialization system 1714 may also provide order-specific data to each module 1510, 1512, 1514, 1516 so they can perform their respective required functions. For instance, serialization system 1714 may transfer specific injection times to dispensing module 1512 to thereby instruct dispensing module 1512 to dispense an RLT composition for injection times that comport with specific customer orders. As another example, serialization system 1714 may transfer data to ILA module 1514 indicating a specific format and data that should be used to comport with specific customer orders during a labeling process executed by ILA module 1514. Serialization system 1714 may similarly provide order-specific label format and data to shipping module 1516. Serialization system 1714 may also collect vessel-specific data for subsequent steps and reporting purposes. For instance, serialization system 1714 may transfer dispensing data for an injection certificate to ERP system 1704. Serialization system 1714 may also transfer batch data to a MES system 1712, which may be part of computing system 1508.

ERP system 1704 also sends batch data to a MES system 1712 in this example. MES system 1712 further sends and receives batch data to and from synthesis module 1510. Similarly, MES system 1712 sends and receives batch data to and from dispensing module 1512.

Synthesis module 1510 also receives data from a recipe source 1710, which may also be part of computing system 1508 or some other system. The data from recipe source 1710 may include recipes for various RLT compositions. As noted above, synthesis module 1510 receives batch data from MES system 1712, may synthesize batch data, and may transfer relevant batch data back to MES system 1712. Synthesis module 1510 also sends batch data to a history repository 1708, which may include a storage device that is part of computing system 1508 or some other system. The data stored in history repository 1708 may include historical manufacturing data from modules 1510, 1512, 1514, 1516.

Dispensing module 1512 also receives data from recipe source 1710. As noted above, dispensing module 1512 receives batch data from MES system 1712 and injection time data from serialization system 1714. Dispensing module 1512 may also scan barcodes of components received in dispensing module 1512 and send the scanned barcode data to serialization system 1714. In addition, dispensing module 1512 may calculate a fill volume for a vessel (e.g., based on an injection time received from serialization system 1714). Dispensing module 1512 may further track other filling activity as the vessel is being filled, then pass the calculated fill volume and data associated with the tracked filing activity to serialization system 1714. Dispensing module 1512 also sends batch data to history repository 1708.

As noted above, ILA module 1514 receives order-specific label format and data from serialization system 1714. Like ILA module 1400 described above, ILA module 1514 of this example may perform inspections of vessels (e.g., vials, syringes, etc.) containing RLT compositions; may print, inspect, and apply labels to such vessels; and may print, inspect, and apply labels to shield assemblies in which the vessels are received. ILA module 1514 may also scan barcodes of components received in dispensing module 1512 and send the scanned barcode data in conjunction with inspection results to serialization system 1714. This may have the effect of tying certain inspection results to certain vessels or shield assemblies, etc. In some cases, ILA module 1514 sends inspection results to serialization system 1714 without necessarily tying those inspection results to certain barcodes. ILA module 1514 also sends batch data to history repository 1708.

As noted above, shipping module 1516 receives order-specific label format and data from serialization system 1714. Shipping module 1516 may also aggregate shield assemblies that contain vessels with RLT compositions into a shipping case. By way of example only, each shipping case may contain one to four shield assemblies or any other suitable number of shield assemblies. In some versions, the shipping case include radiation shielding. In some other versions, the shipping case does not include radiation shielding. Shipping module 1516 may also scan barcodes (or other indicia) on the shield assemblies as the shield assemblies are loaded into the shipping cases. Shipping module 1516 may also print a shipping label and/or any other necessary materials. Shipping module 1516 also sends batch data to history repository 1708.

The data flow 1700 described above is just one illustrative example. Data may flow between the above-described components in various other ways; and various other components may be incorporated into the data flow.

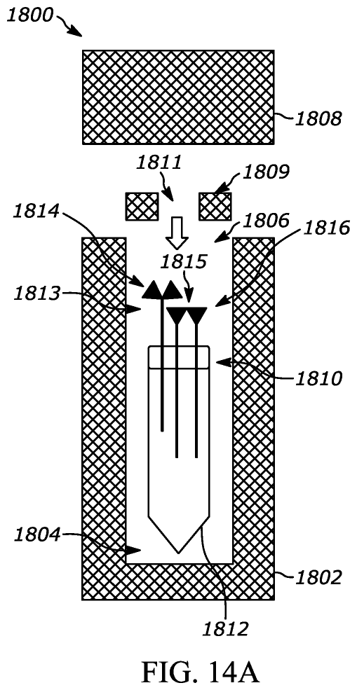

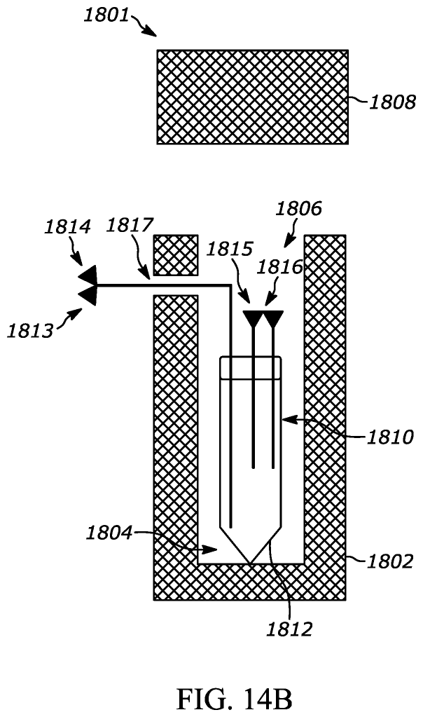

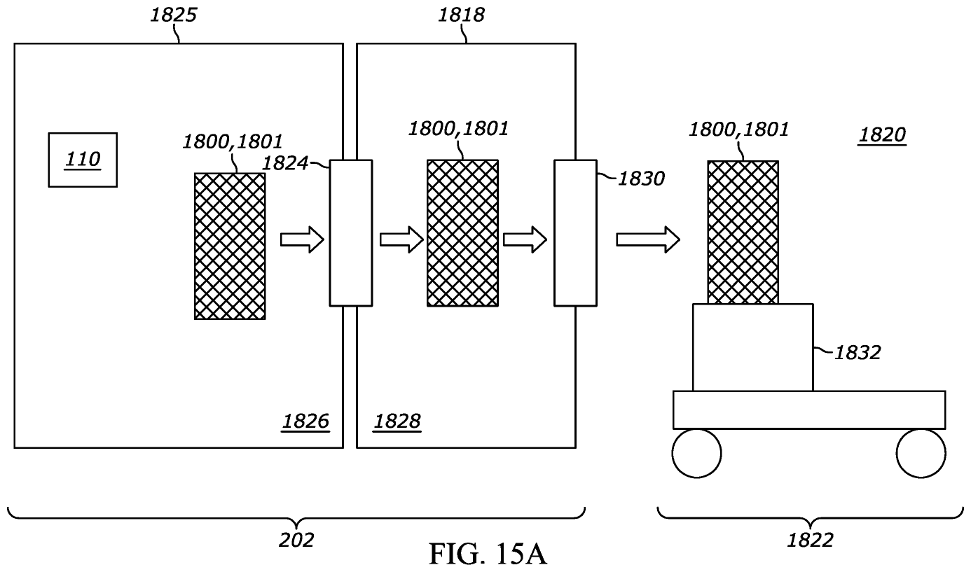

Returning to embodiments of FIG. 2, the radiopharmaceutical composition, produced in the first unit 201 at the synthesis module 110 can be transported to the packaging module 210 of the second unit 202 for further processing. In some embodiments, the radiopharmaceutical composition is transported from the synthesis module 110 to the dispensing module 120. The radiopharmaceutical composition can be transported in a radiation shielded container, which limits or prevents radiation exposure to the operators 150 during transport. Exemplary radiation shielded containers are depicted in FIGS. 14A and 14B.

FIG. 14A depicts a radiation shielded container 1800 in accordance with some embodiments of the present disclosure. The radiation shielded container 1800 can include a container portion 1802 having an interior volume 1804 and a first opening 1806. A lid 1808 can be removably coupled to the container portion 1802 and can cover the first opening 1806 and enclose the interior volume 1804. Alternatively, and not depicted in FIG. 14A, the lid 1808 can be moveable with respect to the container portion 1802, such attached via a hinge or other mechanism. A secondary lid 1809 can rest in the first opening 1806 such that the lid 1808 can cover the secondary lid 1809 and removably attach to the container portion 1802. The secondary lid 1809 can have an opening 1811, where the opening 1811 can be utilized to feed one or more connectors of a bulk solution container (described below) through the opening 1811 from the interior volume 1804. The secondary lid 1809 provides additional radiation shielding, for example, when an operator removes the lid 1808 to facilitate transfer of the radiopharmaceutical composition that resides in the bulk solution container disposed in the interior volume 1804.

The radiation shielded container 1800 can comprise a radiation shielding material. Exemplary radiation shielded materials can include tungsten, sintered tungsten, lead, powder coat or coated with stainless steel. The radiation shielded container 1800 can have a rectangular cross section as shown in FIG. 14A and can have a circular or square footprint. The overall volume occupied by the radiation shield container 1800 can range around 1 liter (L).

The radiation shielded container 1800 can include a bulk solution container 1810 disposed in the interior volume 1804 thereof. The bulk solution container 1810 is utilized to contain the radiopharmaceutical composition during transport between the first unit 201 and the second unit 202. The bulk solution container 1810 can be sized to hold the radiopharmaceutical composition in bulk, for example, where the bulk composition can be dispensed in the second unit 202 into individual vials for packaging and distribution to patients. The bulk solution container can be sized to hold volumes of the radiopharmaceutical composition, for example, capable of filling around 95 individual vials, wherein each vial holds about 7.5 to 12 mL and is around 25-33 mm thick.

The bulk solution container 1810 can include a bulk solution container portion 1812 for holding the radiopharmaceutical composition and one or more connectors coupled to the bulk solution container portion 1812. In this instance, aseptic, non-drip connectors are used for this application. An example of an aseptic connector used for this application may be a CPC AseptiQuik connector. The materials of construction for the aseptic connector can be made from polymers such as polycarbonate and uses seals made from materials such as platinum cured silicone. Connector is certified to be USP VI safe and animal derived, component free (ADCF) prior to use in manufacturing stages or liquid transfer process. The one or more connectors can be utilized as inlets and/or outlets for the bulk solution container portion 1812. The one or more connectors can include an outlet connector 1813, a backup outlet connector 1814, a vent connector 1815, and inlet connector 1816. The one or more connectors can be positioned externally to the bulk solution container portion 1812 as depicted in FIG. 14A. The one or more connectors can be attached to an end of a rigid or flexible tube that extends internally into the bulk solution container portion 1812. Moreover, though depicted in FIG. 14A as being proximate the bulk solution container portion 1812, the one or more connectors may be positioned at an end of a rigid or flexible tube that can extend a substantial distance from the bulk solution container portion 1812, for example, up to 5 lengths of the bulk solution container portion 1812.

The outlet connector 1813 can be used to connect the bulk solution container 1810, and more specifically the bulk solution container portion 1812 which can include the radiopharmaceutical composition, to a second unit 202 as discussed below. The back outlet connector 1814 can be used to perform the same function as the outlet connector 1813. For example, the backup outlet connector 1814 can be used, for example, if the outlet connector 1813 were to fail. For example, in the event of a failure of the outlet connector 1814 and the absence of the backup outlet connector 1814, a entire batch of radiopharmaceutical composition in the bulk solution container 1810 can be wasted due to a lack of a connector to transfer the radiopharmaceutical composition. The vent connector 1815 can be used to allow air flow into the bulk container portion 1812, for example, during transfer of the radiopharmaceutical composition in or out of the bulk solution container portion 1812 The inlet connector 1816 can be used to transfer the radiopharmaceutical composition from the synthesis module 110 in the first unit 201 to the bulk solution container 1810.

The bulk solution container portion 1812 can have a volume ranging from about 0.1 milliliters (ml) to about 2000 milliliters (ml). The bulk solution container portion 1812 can comprise any material compatible with the radiopharmaceutical composition. Exemplary materials can include borosilicate glass or polypropylene. The connectors 1813, 1814, 1815 and/or any flexible and/or rigid tubes connected thereto can include any material compatible with the radiopharmaceutical composition. Exemplary materials can include platinum cured silicon (low impurity extraction silicon) or other polypropylene material.

FIG. 14B depicts a radiation shielded container 1801 in accordance with some embodiments of the present disclosure. The radiation shielded container 1801 is the same as the radiation shielded container 1800, except includes a second opening 1817 disposed in the container portion 1802. The second opening 1817 fluidly couples the interior volume 1804 to an external environment. The second opening 1817 can be smaller than the first opening 1806. For example, as shown in FIG. 14B, the second opening 1817 can be sized to accommodate the outlet and backup outlet connectors 1813, 1814 of the bulk solution container 1810, but not the entire bulk solution container 1810. As depicted in FIG. 14B, the outlet and backup outlet connectors can be disposed at an end of a tube (e.g., flexible or rigid) that can extend through the second opening 1817. In operation, the second opening 1817 can be used to transfer the radiopharmaceutical composition from the bulk solution container 1810 without removing or opening the lid 1808 of the radiation shielded container 1801. The second opening 1817 can further include a gasket or other type of seal (not shown in FIG. 14B) for isolating the interior volume 1804 of the radiation shielded container 1801 when the outlet and backup outlet connectors 1813, 1814 are disposed through the second opening 1817.

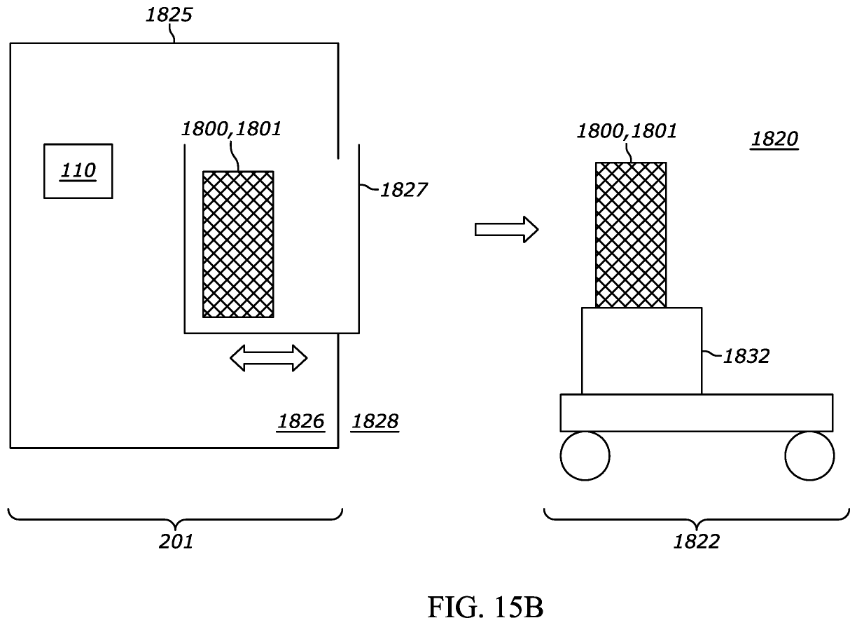

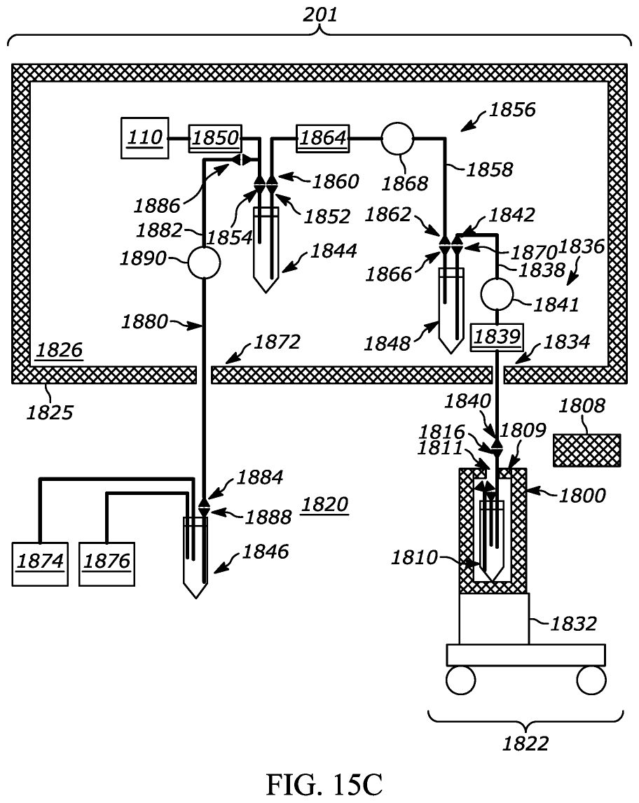

FIGS. 15A to 15B depict a schematic view of the first unit 201 in accordance with some embodiments of the present disclosure. As depicted in FIG. 15A, the first unit 201 can include the synthesis module 110 surrounded by the isolator 1825 and a first decontamination module 1818 coupled thereto, wherein the radiation shielded container 1800, 1801 is loaded with the radiopharmaceutical composition in the interior volume of the isolator 1825. Interior surfaces of the isolator 1825 may have average surface roughness (Ra)≤0.8 μm. Once loaded, the radiation shielded container 1800, 1801 can be transferred to an external environment 1820, e.g. outside of the isolator 1825, outside of the first unit 201. Upon transfer to the external environment, the radiation shielded container 1800, 1801 is loaded onto a transportation machine 1822 and transferred to the second unit 202, e.g., to the dispensing module 120.

In the embodiments of FIG. 15A, the isolator 1825 can be coupled to the decontamination module through a first valve 1824. The first valve 1824, when in an open position, fluidly couples an interior volume 1826 of the isolator 1825 to an interior volume 1828 of the first decontamination module 1818. A second valve 1830 can be disposed in the first decontamination module 1818. The second valve 1830, when in an open position, fluidly couples the interior volume of the first decontamination module 1818 to the external environment 1820.

In operation, the first decontamination module 1818 can be adjusted to have at least the same Grade environment, e.g., A, B, C, D, as the isolator 1825. The second valve 1830 can be in a closed position, and the first valve 1824 can be moved to an open position. Once in the open position, the radiation shielded container 1800, 1801 can be moved from the interior volume 1826 of the isolator 1825 to the interior volume 1828 of the first decontamination module 1818. The first valve 1824 can then be closed. The decontamination module 1818 can then decontaminate the radiation shielded container 1800, 1801. Decontamination methods used in this stage of the process can include, but not be limited to, vaporized hydrogen peroxide decontamination cycles and ionized hydrogen peroxide cycles. Alternatively, if the external environment 1820 is a lower Grade than that of the decontamination module 1818, then a decontamination process cannot be performed. The second valve 1830 can then be opened to the external environment 1820 and the radiation shielded container 1800, 1801 can be removed from the decontamination module 1818 and loaded onto the transport machine 1822 and transferred to the second unit 202, e.g., to the dispensing module 120.

The transport machine 1822 can be any suitable transport machine that can move the radiation shielded container 1800, 1801 to the second unit 202. For example, as shown in FIG. 15A, the transport machine 1822 can be a wheeled cart or trolley. The transport machine 1822 can be motorized or manually pushed. In some embodiments, the transport machine 1822 can be on a rail system (not shown in FIG. 15A). The transport machine 1822 can include a mechanism 1832 for raising and lowering the radiation shielded container 1800, 1801. For example, the mechanism 1832 can raise to the level of the second valve 1830 to receive the radiation shielded container 1800, 1801. The mechanism 1832 can then lower with the radiation shielded container 1800, 1801 prior to transport to the second unit 201. The mechanism can be manual or automated.

In the embodiments of FIG. 15B, isolator 1825 may include a drawer 1827 for transfer of the radiation shielded container 1800, 1801 (instead of the decontamination module 1818 as depicted in FIG. 15A). The drawer 1827 can extend through an opening in the isolator 1825 from the interior volume 1826 of the isolator 1825 to the external environment 1820. In operation, the radiation shielded container 1800,1801 can be loaded into the drawing 1827 in the interior volume 1826 while the drawer 1827 is closed to the exterior environment 1820. Then the drawer 1827 can be moved laterally to close the drawer 1827 to the interior volume 1826 and open the drawer 1827 to the external environment 1820. The radiation shieled container 1800, 1801 can be placed on the transportation machine 1822 and transferred to the second unit 202, e.g., to the dispensing module 120.

In the embodiments of FIG. 15C, the radiation shielded container 1800, 1801 (container 1801 is shown in FIG. 15C) can be loaded in the external environment 1820. The isolator 1825 can include an opening 1834, where the opening includes a first kit 1836 extending therethrough from the interior volume 1826 of the isolator 1825 to the external environment 1820. Optionally, a gasket or other sealing device (not shown) can be disposed in the opening to seal the opening 1834 such that the interior volume 1826 of the isolator 1825 is not fluidly coupled to the external environment 1820. In some embodiments, the interior volume 1826 and the external environment 1820 can be Grade C environments where no seal is needed between the interior volume 1826 and external environment 1820.

The first kit 1836 can include a tube 1838 having a first connector 1840 and a second connector 1842 at opposing ends thereof. A filter 1839 can be disposed inline with the tube 1838 between the first and second connectors 1840, 1842 and used to filter the radiopharmaceutical composition prior to entering the bulk solution container 1810. The first connector 1840 can be coupled to the inlet connector 1816 of the bulk solution container 1810, and the first kit 1836 can facilitate transfer of the radiopharmaceutical composition from the first unit 201 into the bulk solution container 1810. A peristaltic pump 1841 can be coupled to the tube 1838 to facilitate movement of the radiopharmaceutical composition to the bulk solution container 1810.

In operation, the radiation shielded container 1800 (or container 1801—not depicted in FIG. 15C) having the bulk solution container 1810 disposed therein can be disposed on the transportation machine 1822 and in the external environment 1820, i.e., external to the interior volume 1826 of the isolator 1825. To facilitate a connection of the first kit 1836 to the bulk solution container 1810, the lid 1808 can be removed from the radiation shielded container 1800, the inlet connector 1816 of the bulk solution container 1818 can be positioned through the opening 1811 in the secondary lid 1809, and the second connector 1842 of the first kit 1836 can be coupled to the inlet connector 1816 of the bulk solution container 1810.

FIG. 15C further depicts connectivity between the synthesis module 110 and the first kit 1836 in accordance with some embodiments of the present disclosure. The synthesis module 110 can produce a concentrated radiopharmaceutical composition which can then be diluted to a desired radioactive concentration. As depicted in FIG. 15 C, in some embodiments of the present disclosure, the synthesis module 110 can be coupled to a mother vial 1844, where the mother vial can receive the concentrated radiopharmaceutical composition from the synthesis module 110 and separately can receive a dilution solution from a dilution container 1846. The radioactive concentration of contents of the mother vial 1844 can be measured until the desired radiopharmaceutical composition with the desired radioactive concentration is reached. Once the desired radiopharmaceutical composition is achieved, the composition can be transported from the mother vial 1844 to a mixing container 1848 to ensure homogeneity of the radiopharmaceutical composition. The mixing container 1848 can be coupled to the bulk solution container 1810 through the first kit 1836.

The synthesis module 110 can be coupled to the mother vial 1844 via a filter 1850. The filter 1850 may be any suitable filter for removing contaminants from the concentrated radiopharmaceutical composition. Contaminants can include one or more common waterborne and environmental bacteria such as Pseudomonas aeruginosa (ATCC 9027), Ralstonia pickettii (ATCC 49129), and Burkholderia cepacia complex (ATCC 25416), along with Gram-positive cleanroom and raw-material contaminants like Bacillus subtilis (ATCC 6633), Bacillus cereus (ATCC 14579), and skin-associated cocci such as Staphylococcus aureus (ATCC 6538) and Micrococcus luteus (ATCC 10240). These organisms represent the typical bioburden flora that membrane filtration (0.45 μm or 0.22 μm) is designed to capture before downstream sterile processing. Exemplary filters can include Millipak® filters, available from Merck KGaA of Darmstadt, Germany, and Pall™ (Cytiva) Supor™ filters of Wilmington, DE, United States.

The mother vial 1844 can include an inlet connector 1852 and an outlet connector 1854 in accordance with some embodiments of the present disclosure. The mother vial 1844 can include a vent connector (not depicted in FIG. 15C). The mother vial 1844 can be connected to the mixing container 1848 through a second kit 1856. The second kit 1856 can include a tube 1858 having a first connector 1860 and a second connector 1862. The tube 1858 can include a filter 1864 that can be in-line between the first connector 1860 and the second connector 1862. The filter 1864 can be any filter as described above with reference to filter 1850. The first connector 1860 can be coupled to the outlet connector 1854 of the mother vial 1844, and the second connector 1862 can be connected to an inlet connector 1866 of the mixing container 1848. A peristaltic pump 1868 can be coupled to the tube 1858 of the second kit 1856 to facilitate movement of the radiopharmaceutical composition from the mother vial 1844 to the mixing container 1848. Once homogenized in the mixing container, the radiopharmaceutical composition can exit the mixing container 1848 via an outlet connector 1870, the outlet connector can be coupled to the second connector 1842 of the first kit 1836.

Returning to the inlet side of the mother vial 1844, the dilution container 1846 can be located external to the isolator 1825 and accessible via an opening 1872 in the isolator 1825. The dilution container 1846 can be located in the external environment 1820, or in another environment (not shown in FIG. 15C) separated from the external environment 1820 and from the interior volume 1826 of the isolator 1825. The dilution container 1846 can be coupled to solvent container 1874 and an additive container 1876. The solvent container 1874, for example, can supply a solvent, such as water, to the dilution container. The additive container 1876, for example, can supply an additive, such as one or more a sequestering agent, a stabilizer, an isotonic agent, a pH adjusting agent, and the like. The dilution container 1846 can be coupled to the mother vial 1844 via a third kit 1880. The third kit 1880 can include a tube 1882 having a first connector 1884 and second connector 1886. The first connector 1884 can be coupled to an outlet connector 1888 of the dilution container 1846 and the second connector 1886 can be coupled directly (not shown in FIG. 15C) or indirectly to the inlet connector 1852 of the mother vial 1844. A peristaltic pump 1890 can be coupled to the tube 1882 of the third kit 1880 to facilitate transfer of a dilution solution from the dilution container 1846 to the mother vial 1844.

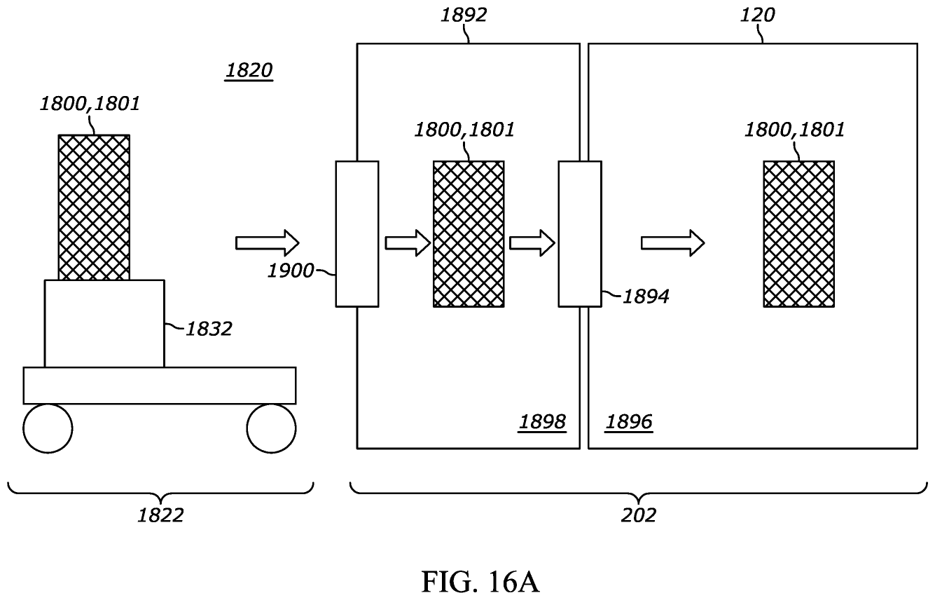

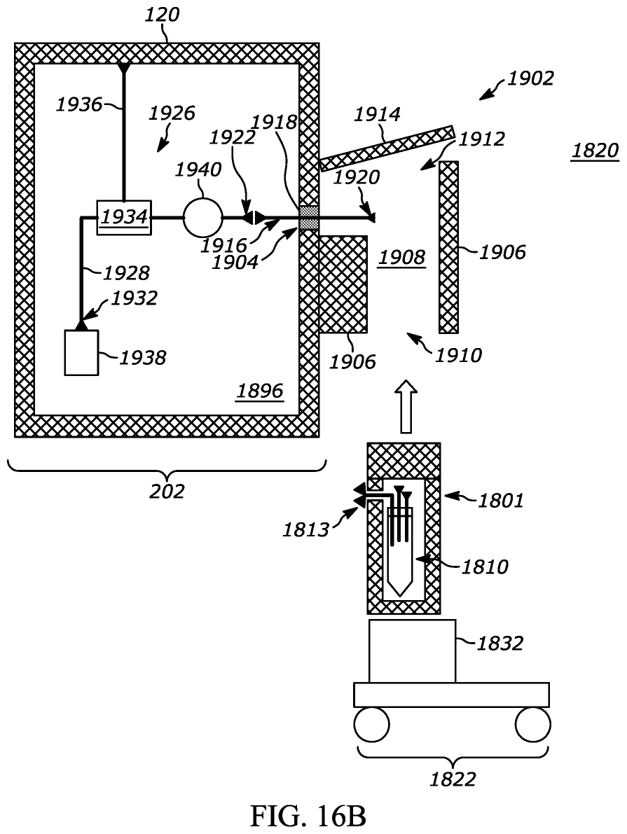

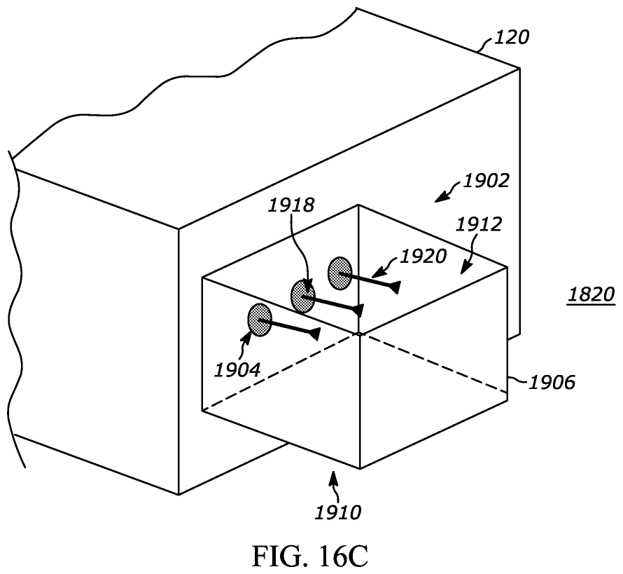

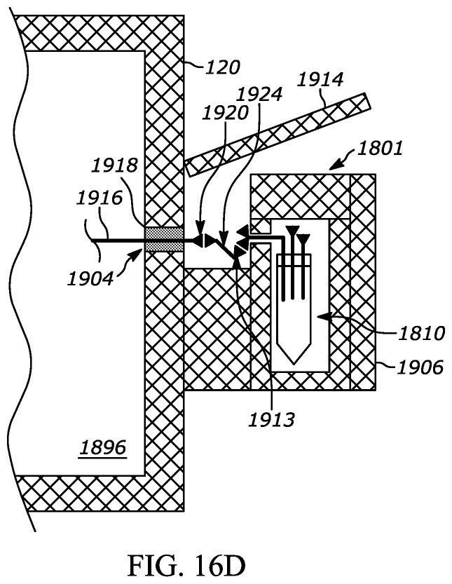

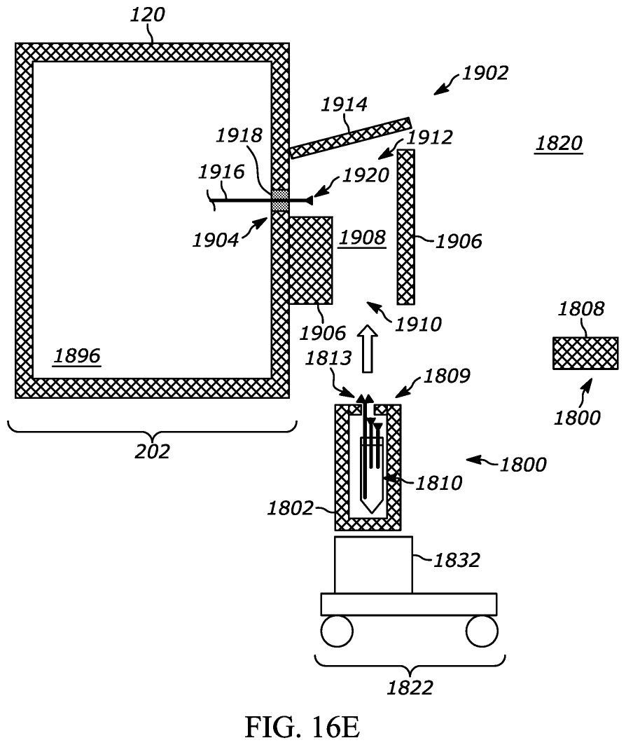

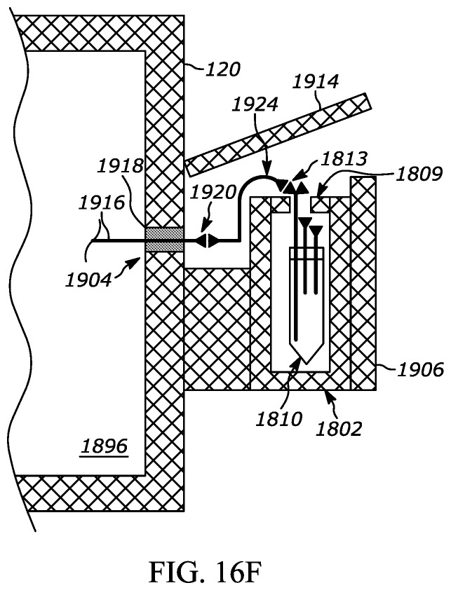

FIG. 16A depicts a second unit 202 and the transport machine 1822 in accordance with some embodiments of the present disclosure. Once the transport machine 1822 arrives at the second unit 201, the radiopharmaceutical composition can be unloaded into the second unit 202. For example, the unloading can occur by transferring the radiation shielded container into an interior volume of the second unit 202, e.g., into an interior volume of the dispensing module 120 (depicted in FIG. 16A), or the unloading can occur by transferring the radiopharmaceutical composition into the interior volume of the second unit while the radiation shielded container 1800, 1801 and the bulk solution container 1810 remain in the external environment 1820, e.g., external to the interior volume of the second unit 202 (depicted in FIGS. 16 B to 16E).

In the embodiments of FIG. 16A, the second unit 202 includes the dispensing module 120 and the dispensing module 120 can be coupled to a second decontamination module 1892 through a third valve 1894. The third valve 1894, when in an open position, fluidly couples an interior volume 1896 of the dispensing module 120 to an interior volume 1898 of the second decontamination module 1838. A fourth valve 1900 can be disposed in the second decontamination module 1892. The fourth valve, when in an open position, fluidly couples the interior volume 1898 of the second decontamination module 1892 to the external environment 1820.

In operation, the third valve 1894 can be closed to isolate the interior volume 1896 of the dispensing module 120. The fourth valve 1900 can then be opened and the radiation shielded container 1800, 1801 can be transferred into the interior volume 1898 of the second decontamination module 1892. The radiation shielded container 1800, 1801 can be transferred manually or by an automated process, for example, by a transfer mechanism located on the transport machine 1822 or in the external environment 1820 that transfers the radiation shielded container 1800, 1801 into the second decontamination module 1892. Once the radiation shielded container 1800, 1801 is inside the second decontamination module 1892, the fourth valve 1900 can be closed. The second decontamination module 1892 can be adjusted to have at least the same Grade environment, e.g., A, B, C, D, as the dispensing module 120. The second decontamination module 1892 can then decontaminate the radiation shielded container 1800, 1801. Decontamination methods used in this stage of the process can include, but not be limited to, vapourised hydrogen peroxide decontamination cycles and ionized hydrogen peroxide cycles. Once decontaminated, the third valve 1894 can be opened and the radiation shielded container 1800, 1801 can be transferred into the interior volume 1896 of the dispensing module 120. The third valve 1894 can be closed once the transfer is complete. Once inside the dispensing module 120, the bulk solution container 1810 can be coupled to a transfer kit as discussed below to transfer the radiopharmaceutical composition from the bulk solution container into individual packaging containers, such as vials, syringes, or the like.

In the embodiments of FIGS. 16B to 16D, the radiation shielded container 1801 can be unloaded to the dispensing module 120 from the external environment 1820. The dispensing module 110 can include a radiation shielded pocket 1902 where the radiation shielded pocket 1902 is configured to interface with the radiation shielded container 1801. The radiation shielded pocket 1902 can cover an opening 1904, wherein the opening 1904 is configured to receive the radiopharmaceutical composition from the bulk solution container 1810.

The radiation shielded pocket 1902 can include a housing 1906 having an interior volume 1908. The interior volume 1854 can be fluidly coupled to a lower opening 1856 and an upper opening 1858. The opening 1904 can be fluidly coupled to the interior volume 1908 of the housing 1906 between the upper and lower openings 1910, 1912. The pocket 1902 includes a lid 1914 which can be coupled to the housing 1906 and moveable with respect thereto. The lid 1914 can have an open position (as shown in FIGS. 16B to 16C) and a closed position (not shown), where the closed position of the lid 1914 covers the upper opening 1912. The radiation shielded container 1801 is configured to enter the housing 1906 through the lower opening 1910.

The opening 1904 includes a tube 1916 (e.g., a flexible or rigid tube) extending therethrough from the interior volume 1896 of the dispensing module 120 to the external volume 1820. A gasket 1918 or other sealing device can be disposed in the opening to seal the opening 1904 such that the interior volume 1896 of the dispensing module 120 is not fluidly coupled to the external environment 1820. Further, and depicted in FIG. 16C (where the lid 1914 is omitted from this view), a plurality of openings 1904 can be disposed in the radiation shieled pocket 1902, where each opening 1904 includes a tube 1916 extending therethrough from the interior volume 1896 of the dispensing module 120 to the external volume 1820. Each opening 1904 can include a gasket 1918 or other sealing device. A plurality of openings 1904 can be utilized to reduce the frequency of decontamination processes. For example, for each batch (each bulk solution container 1810 holding one batch of radiopharmaceutical composition) one transfer kit (described below) and one tube 1916 are used. After the batch is transferred to the interior volume 1896 and processed, the transfer kit and tube 1916 would be replaced. The replacement involves opening the dispensing module 120 to the external environment, replacing the tube 1916 and replacing the transfer kit. The dispensing module 120 is then closed and decontaminated before proceeding to the next batch. When a plurality of tubes 1916 are used, one at each opening 1904, each tube 1916 can be used for one batch, and then the dispensing module 120 can be opened to the external environment and all of the plurality of tubes 1916 can be replaced simultaneously and the dispensing module 120 can then be decontaminated. Thus, frequency of decontamination processes can be reduced by having a plurality of openings 1904 and tubes 1916. Further, multiple transfer kits can be disposed in the interior volume 1896, where one transfer kit is used for each batch. During the opening and decontamination of the dispensing module 120, the multiple transfer kits can be replaced with fresh transfer kits.

In operation, the radiation shielded container 1801 can be inserted into the radiation shielded pocket 1902 through the lower opening 1910. The container 1801 can be inserted, for example, by raising the container 1801 using the mechanism 1832 on the transport machine 1822. The tube 1916 can included a first connector 1920 and second connector 1922 at opposing ends thereof. The first connector 1920 of the tube 1916 can be connected to the outlet connector 1813 (or the backup outlet connector 1814 if necessary) of the bulk solution container 1810 and the radiopharmaceutical composition can be transferred from the bulk solution container portion 1812 through the tube 1916 and into the interior volume 1896 of the dispensing module 120. The bulk solution container 1810 can be connected directly to the tube 1916 (not shown in FIGS. 16B to 16D), or connected through an auxiliary tube 1924, where the auxiliary tube 1924 extends between the outlet connector 1813 and the first connector 1920. To connect the bulk solution container 1810 to the tube 1916, the lid 1914 can be moved to the open position (as shown in FIG. 16D) such that both the outlet connector 1813 and the tube 1916 are accessible to make the connection.

As depicted in FIGS. 16E to 16F, a similar transfer of the radiopharmaceutical composition can be performed using the radiation shielded container 1800, except the lid 1808 would be removed prior to inserting the radiation shielded container portion 1802 and secondary lid 1809 into the pocket 1902 to permit connection of the bulk solution container 1810 to the tube 1916.

The tube 1916 can be coupled to a transfer kit 1926 in accordance with some embodiments of the present disclosure and depicted in FIG. 16B. The transfer kit 1926 includes a tube 1928 (e.g., flexible or rigid) having a first connector 1930 and a second connector 1932. The transfer kit 1926 can included a filter 1934 disposed in-line with the tube 1926. The filter 1934 can be vented through an auxiliary line 1936 which can vent externally from the interior volume 1896 of the dispensing module 120. The first connector 1930 can be coupled to the second end of the tube 1916 and the second connector 1932 can be coupled to a single dose vial 1938. A peristaltic pump 1940 can be coupled to the tube 1928 to facilitate transfer of the radiopharmaceutical composition from the bulk solution container 1810 to a single dose vial 1938.

In operation, the radiopharmaceutical composition can be transferred from the bulk solution container 1810 through the tube 1928 of the transfer kit 1926 using the peristaltic pump 1940. The radiopharmaceutical composition passes through the filter 1934 prior to exiting the transfer kit 1926 at the second connector 1932.

The transfer kit 1926 can transfer a single dose of the radiopharmaceutical composition at a time from the bulk solution container 1810 into one single dose vial 1938. Post filling of the packaging container, the single dose vial 1938 can be sealed, scanned and coded, and then placed in a first shielded container, the first shielded container being open. The single dose vial 1938 in the open shielded container can further be inspected, labeled and transferred into a second shielded container, the second shielded container then being sealed and shipped. The first shielded container can be reused.

The transfer kit 1900 can fill, for example, up to about 2 single dose vials 1938 per minute from the bulk solution container 1810. Once the bulk solution container 1810 has been emptied of the radio pharmaceutical composition, the transfer kit 1926 is replaced with a new transfer kit 1926 for the next bulk solution container 1810. The replacement process can include opening the dispensing module 120, removing and replacing the transfer kit 1926, cleaning the and closing the dispensing module 120.

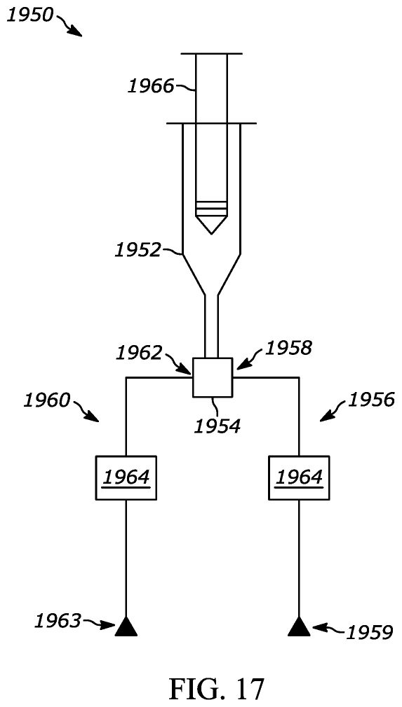

FIG. 17 depicts a transfer kit 1950 in accordance with some embodiments of the present disclosure. The transfer kit 1950 includes a syringe 1952 and a valve 1954 coupled to the syringe 1952. The transfer kit 1950 further includes a first rigid tube 1956 having a first end 1958 coupled to the valve 1954 and a second rigid tube 1960 having a first end 1962 coupled to the valve 1954. The valve 1954 can have a first open position where the first rigid tube is open to the syringe 1952 and the second rigid tube 1960 is closed to the syringe 1952, and a second open position where the first rigid tube 1956 is closed to the syringe 1952 and the second rigid tube 1960 is open to the syringe 1952. The first rigid tube 1956 can be coupled to the outlet connector 1813 of the bulk solution container 1810, for example, when the bulk solution container 1810 is disposed with the interior volume 1896 of the dispensing module 120 as depicted in FIG. 16A. The transfer kit 1950 can include one or more filters 1964 where the one or more filters 1918 are dispose in-line in at least one of the first or second rigid tubes 1956, 1960. As shown in FIG. 17, and in some embodiments, the kit 1950 has two filters 1964 where one filter 1964 is disposed in-line in the first rigid tube 1908 and the other filter 1964 is disposed in-line in the second rigid tube 1960.

In operation, a second end 1959 of the first rigid tube 1956 can be coupled to the outlet connector 1813 of the bulk solution container 1810. The valve 1954 can be set in the first open position where the first rigid tube 1956 is open to the syringe 1952. The radiopharmaceutical composition can be transferred from the bulk solution container portion 1812 into the syringe 1952 by pulling a plunger 1966 of the syringe 1952. The valve 1954 can then be set to the second open position where the syringe 1952 is open to the second rigid tube 1960. The plunger 1966 can then be pushed to move the radiopharmaceutical composition from the syringe 1952 into the second rigid tube 1960 and ultimately to the single dose vial 1938 through a second end 1963 of the second rigid tube 1960. The radiopharmaceutical composition passes through the one or more filters 1964 prior to exiting the transfer kit 1950 at the second end 1963 of the second rigid tube 1960.

### ADDITIONAL NOTES

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The above-described implementations described herein can be implemented in any of numerous ways. For example, some implementations may be implemented using hardware, software or a combination thereof. When any aspect of an implementation is implemented at least in part in software, the software code can be executed on any suitable processor or collection of processors, whether provided in a single computer or distributed among multiple computers.

In this respect, various aspects of the implementations described herein may be implemented at least in part as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various implementations of the technology discussed above. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this implementation, one or more computer programs that when executed perform methods of the present technology need not reside on a single computer or processor, but may be distributed in a modular fashion amongst a number of different computers or processors to implement various aspects of the present technology.

Also, the technology described herein may be implemented as a method, of which at least one example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

All definitions, as defined and used herein, should be understood to control over dictionary definitions, definitions in documents incorporated by reference, and/or ordinary meanings of the defined terms.

To the extent applicable, the terms “first,” “second,” “third,” etc. herein are merely employed to show the respective objects described by these terms as separate entities and are not meant to connote a sense of chronological order, unless stated explicitly otherwise herein.

The indefinite articles “a” and “an,” as used herein in the specification and in the claims, unless clearly indicated to the contrary, should be understood to mean “at least one.” Any ranges cited herein are inclusive.

The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. In some instances, these terms include no fluctuations—e.g., 0%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one implementation, to A only (optionally including elements other than B); in another implementation, to B only (optionally including elements other than A); in yet another implementation, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one implementation, to at least one, optionally including more than one, A, with no B present (and optionally including elements other than B); in another implementation, to at least one, optionally including more than one, B, with no A present (and optionally including elements other than A); in yet another implementation, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A modular manufacturing system, comprising:
a first unit to synthesize a radiopharmaceutical composition; a second unit to package the radiopharmaceutical composition into a radiopharmaceutical product; and
a transport machine configured to transport the radiopharmaceutical composition from the first unit to the second unit, wherein at least a portion of the second unit has a higher grade manufacturing environment than at least a portion of the first unit.

2. The modular manufacturing system of claim 1, wherein at least a portion of the first unit has a Grade D environment, and wherein at least a portion of the second unit has a Grade C environment.

3. The modular manufacturing system of claim 1, wherein the first unit further comprising:
an isolator having an interior volume; and a synthesis module disposed in the interior volume, wherein the synthesis module is configured for producing a concentrated radiopharmaceutical composition.

4. The modular manufacturing system of claim 3, wherein the interior volume of the isolator is a Grade D environment.

5. The modular manufacturing system of claim 3, wherein the first unit further comprises:
a mother vial;
a dilution container configured to hold a dilution solution; and
a mixing container,
wherein the mother vial is coupled to the synthesis module to receive the concentrated radiopharmaceutical composition and coupled to the dilution container to receive the dilution solution, wherein the mother vial is further coupled to the mixing container, and
wherein the mixing container is configured to receive a mixture of the concentrated radiopharmaceutical solution and the dilution solution from the mother vial and to homogenize the mixture into the radiopharmaceutical composition.

6. The modular manufacturing system of claim 5, the isolator having a first opening and a second opening.

7. The modular manufacturing system of claim 6, wherein the dilution container is disposed external to the interior volume of the isolator and coupled to the mother vial through the first opening.

8. The modular manufacturing system of claim 6, further comprising:
a radiation shielded container having a bulk solution container disposed therein,
wherein the bulk solution container configured to contain the radiopharmaceutical composition, wherein the transport machine having the radiation shielded container disposed thereon, and
wherein the radiation shielded container and transport machine are disposed external to the interior volume of the isolator.

9. The modular manufacturing system of claim 8, wherein the transport machine further comprises a mechanism for raising and lowering the radiation shielded container.

10. The modular manufacturing system of claim 8, wherein the bulk solution container is coupled to the mixing container through the second opening.

11. The modular manufacturing system of claim 10, wherein the radiation shielded container comprises:
a container portion having an interior volume and a first opening; and
a lid removably coupled to the container portion to cover the first opening and enclose the interior volume,
wherein the bulk solution container is disposed in the interior volume.

12. The modular manufacturing system of claim 11, wherein the bulk solution container further comprises:
a bulk solution container portion for holding the radiopharmaceutical composition;
an outlet connector coupled to the bulk solution container portion; and
an inlet connector coupled to the bulk solution container portion,
wherein the inlet connector is configured to be coupled to the mixing container via the second opening of the isolator.

13. The modular manufacturing system of claim 12, wherein the bulk solution container further comprises:
a backup outlet connector coupled to the bulk solution container portion; and
a vent connector coupled to the bulk solution container portion.

14. The modular manufacturing system of claim 12, wherein the radiation shielded container further comprising:
a secondary lid covering the first opening and disposed between the lid and the container portion, wherein the secondary lid comprises a third opening fluidly coupled to the interior volume of the container portion, wherein the outlet connector of the bulk solution container is configured to be accessible through the third opening.

15. The modular manufacturing system of claim 12, wherein the second unit further comprises:
a dispensing module having a first interior volume.

16. The modular manufacturing system of claim 15, wherein the first interior volume of the dispensing module is a Grade C environment.

17. The modular manufacturing system of claim 15, wherein the dispensing module further comprises:
a radiation shielded pocket disposed externally from the first interior volume of the dispensing module,
wherein the radiation shielded pocket is configured to interface with the radiation shielded container,
wherein the radiation shielded pocket covers one or more openings in the dispensing module,
wherein the one or more openings extend from the first interior volume of the dispensing module to the radiation shielded pocket, and
wherein the one or more openings is configured to receive the radiopharmaceutical composition from the bulk solution container.

18. The modular manufacturing system of claim 17, wherein the radiation shielded pocket further comprises:
a housing having a second interior volume, where the second interior volume of the housing is fluidly coupled to a lower opening and an upper opening, wherein the one or more openings of the dispensing module are disposed between the lower and upper openings; and
a lid coupled to the housing and movable between an open position and a closed position, wherein the lid covers the upper opening when in a closed position, and
wherein the radiation shielded container is configured to enter the second interior volume of the housing through the lower opening.

19. The modular manufacturing system of claim 17, wherein each of the one or more openings of the dispensing module further comprise:
a first tube disposed therethrough from the first interior volume of the dispensing module to the second interior volume of the housing;
a first connector coupled to one end of the first tube disposed in the second interior volume;
a second connector coupled to an opposing end of the first tube disposed in the first interior volume; and
a sealing device dispose in the opening such that the first interior volume is not fluidly coupled to the second interior volume.

20. The modular manufacturing system of claim 19, further comprising:
a transfer kit for transferring the radiopharmaceutical composition from the bulk solution container into the first interior volume of the dispensing module via the one or more openings.

21. The modular manufacturing system of claim 20, wherein the transfer kit further comprises:
a second tube having a first connector at one end of the second tube and a second connector at an opposing end of the second tube, wherein the first connector of the second tube is configured to be coupled to second connector of the first tube.

22. The modular manufacturing system of claim 21, wherein the transfer kit further comprises:
one or more filters for decontaminating the radiopharmaceutical composition upon exit from the bulk solution container, wherein the one more filters are disposed in-line in the second tube.

23. The modular manufacturing system of claim 21, further comprising:
an auxiliary line coupled to a vent connector disposed on the one or more filters.

24. The modular manufacturing system of claim 21, further comprising:
a peristaltic pump coupled to the second tube of the transfer kit to facilitate movement of the radiopharmaceutical composition therethrough.

25. The modular manufacturing system of claim 19, wherein the first connector of the first tube is configured to be coupled to the outlet connector of the bulk solution container.
